(19) United States
(12) Patent Application Publication
(10) Pub. No.: US 2026/0287763 A1
(43) Pub. Date: Sep. 24, 2026

(54) DETECTOR BLOCK FOR TOMOGRAPHY SCANNERS

(71) Applicant: Cornell University, Ithaca, NY (US)

(72) Inventors: Amirhossein Goldan, E. Setauket, NY (US); Xinjie Zeng, Long Island City, NY (US); Zipai Wang, Ithaca, NY (US)

(73) Assignee: Cornell University, Ithaca, NY (US)

(21) Appl. No.: 19/476,085

(22) PCT Filed: Apr. 19, 2024
(86) PCT No.: PCT/US2024/025471

§ 371 Date: Oct. 16, 2025

Related U.S. Application Data
(60) Provisional application No. 63/461,082, filed on Apr. 21, 2023.

(51) Int. Cl. G01T 1/20; A61B 6/42; G01T 1/29

(52) U.S. Cl. CPC G01T 1/20187; G01T 1/2985; A61B 6/4233

(57) ABSTRACT

Presented herein are devices for detected photons for tomography. The device can include a plurality of scintillator arrays. Each of the plurality of scintillator arrays can be configured to emit photons in response to receipt of ionizing radiation. Each respective scintillator array of the plurality of scintillator arrays can include a first region corresponding to a first end to an intermedial section of the respective scintillator array. The intermedial section can have a first cross-sectional dimension. Each respective scintillator array of the plurality of scintillator arrays can include a second region corresponding to the intermedial section to a second end of the respective scintillator array. The device can include a light guide coupled with the first end of each of the plurality of scintillator arrays. The device can include a photodetector coupled with the second end of the second scintillator array.

### CROSS-REFERENCE TO RELATED PATENT APPLICATIONS

This application claims priority from Provisional Application U.S. Application 63/461,082, titled “Detector Block for Tomography Scanners,” filed Apr. 21, 2023, which is incorporated herein by reference in its entirety.

### STATEMENT OF GOVERNMENT SUPPORT

The invention was made with government support under R01EB030413 awarded by the National Institute of Health (NIH). The government has certain rights to the invention.

### BACKGROUND

A tomograph may scan an object by applying a signal or wave (e.g., an electromagnetic (EM) wave or a particle emission) to generate a tomogram.

### SUMMARY

At least one aspect is directed to a device for detected photons for tomography. The device can include a plurality of scintillator arrays. Each of the plurality of scintillator arrays can be configured to emit photons in response to receipt of ionizing radiation. Each respective scintillator array of the plurality of scintillator arrays can include a first region corresponding to a first end to an intermedial section of the respective scintillator array. The intermedial section can have a first cross-sectional dimension. Each respective scintillator array of the plurality of scintillator arrays can include a second region corresponding to the intermedial section to a second end of the respective scintillator array. The second end can have a second cross-sectional dimension smaller than the first cross-sectional dimension. The device can include a light guide coupled with the first end of each of the plurality of scintillator arrays. The light guide can be configured to transfer the photons from a first scintillator array to a second scintillator array of the plurality of scintillator arrays. The device can include a photodetector coupled with the second end of the second scintillator array. The photodetector can be configured to convert the photons received via the second scintillator array into an electrical signal for tomography imaging.

In some embodiments, the first region of at least one of the plurality of scintillator arrays can include the first end can have a third cross-sectional dimension smaller than the first cross-sectional dimension of the intermedial section. In some embodiments, the second region of at least one of the plurality of scintillator arrays can have a third cross-sectional dimension tapering from the first cross-sectional dimension at the intermedial section to a third cross-sectional dimension at the second end, to minimize leakage of photons to a second photodetector neighboring the photodetector. In some embodiments, at least one of the plurality of scintillator arrays can include a cuboid structure formed from at least one of a lutetium-based crystal, alkali halide crystal, or an inorganic non-alkali crystal.

In some embodiments, the photodetector has a third cross-sectional dimension corresponding a fourth cross-sectional dimension defined by the second cross-sectional dimension of at least two scintillator arrays of the plurality of scintillator arrays, to localize distribution of photons from the at least two scintillator arrays of the plurality of scintillator arrays. In some embodiments, the photodetector can be configured to send the electrical signal to a circuit. In some embodiments, the circuit can be configured to perform at least one of time-of-flight (ToF) or depth-of-interaction (DOI) using the electrical signal for tomography imaging. In some embodiments, the light guide further can include a prism structure configured to direct photons from the first scintillator array to the second scintillator array of the plurality of scintillator arrays.

At least one aspect is directed to a system. The system can include a block structure. The block structure can include a first layer, a second layer, and a third layer. The block structure can have a plurality of scintillator arrays arranged along the first layer. Each respective scintillator array of the plurality of scintillator arrays can include a first region corresponding to a first end to an intermedial section of the respective scintillator array. The intermedial section can have a first cross-sectional dimension. Each respective scintillator array of the plurality of scintillator arrays can include a second region corresponding to the intermedial section to a second end of the respective scintillator array. The second end can have a second cross-sectional dimension smaller than the first cross-sectional dimension. The block structure can include a plurality of light guides arranged along the second layer. Each of the plurality of light guides can be coupled with at least two of the plurality of scintillator arrays. The block structure can include a plurality of photodetectors arranged along the third layer. Each of the plurality of photodetectors coupled with at least two of the plurality of scintillator arrays.

In some embodiments, the block structure can include an interior region. In some embodiments, at least one of the plurality of light guides in the interior region is coupled with a subset of scintillator arrays including at least four of the plurality of scintillator arrays in the interior region. In some embodiments, at least one of the plurality of photodetectors in the interior region comprises a prism structure configured to transfer photons from at least one of the subset of scintillator arrays to a remainder of the subset of scintillator arrays. In some embodiments, the block structure can include an edge region. The at least one of the plurality of light guides in the edge region is coupled with a subset of scintillator arrays including a first scintillator array and a second scintillator array of the plurality of scintillator arrays in the edge region. In some embodiments, at least one of the plurality of photodetectors in the edge region comprises a prism structure configured to transfer photons from the first scintillator array to the second scintillator array.

In some embodiments, the block structure can include a corner region. In some embodiments, at least one of the plurality of light guides in the corner region is coupled with a subset of scintillator arrays including at least three of the plurality of scintillator arrays in the corner region. In some embodiments, at least one of the plurality of photodetectors in the corner region comprises a prism structure configured to transfer photons from a scintillator array from the subset of scintillator arrays on a corner to a remainder of the subset of scintillator arrays. In some embodiments, the first region of at least one of the plurality of scintillator arrays can include the first end having a fourth cross-sectional dimension smaller than the first cross-sectional dimension of the intermedial section. In some embodiments, the second region of at least one of the plurality of scintillator arrays has a third cross-sectional dimension tapering from the first cross-sectional dimension at the intermedial section to a third cross-sectional dimension at the second end, to minimize leakage of photons to a second photodetector neighboring the photodetector.

In some embodiments, the block structure can include a circuit coupled with the plurality of photodetectors. In some embodiments, the circuit configured to perform at least one of time-of-flight (ToF) or depth-of-interaction (DOI) using a plurality of electrical signals from the plurality of photodetectors.

At least one aspect is directed to a method. The method can include positioning, along a first layer of a block structure, a plurality of scintillator arrays. Each respective scintillator array of the plurality of scintillator arrays can include a first region corresponding to a first end to an intermedial section of the respective scintillator array. The intermedial section can have a first cross-sectional dimension. Each respective scintillator array of the plurality of scintillator arrays can include a second region corresponding to the intermedial section to a second end of the respective scintillator array. The second end can have a second cross-sectional dimension smaller than the first cross-sectional dimension. The method can include positioning, along a second layer of the block structure, a plurality of light guides. Each of the plurality of light guides can be coupled with at least two of the plurality of scintillator arrays. The method can include arranging, along a third layer of the block structure, a plurality of photodetectors. Each of the plurality of photodetectors coupled with at least two of the plurality of scintillator arrays.

In some embodiments, the method can include forming the first region of at least one of the plurality of scintillator arrays to have the first end with a third cross-sectional dimension smaller than the first cross-sectional dimension of the intermedial section. In some embodiments, the method can include forming the second region of at least one of the plurality of scintillator arrays to have a third cross-sectional dimension tapering from the first cross-sectional dimension at the intermedial section to a third cross-sectional dimension at the second end.

In some embodiments, the block structure can include an interior region. In some embodiments, at least one of the plurality of light guides in the interior region is coupled with a subset of scintillator arrays including at least four of the plurality of scintillator arrays in the interior region. In some embodiments, at least one of the plurality of photodetectors in the interior region comprises a prism structure configured to transfer photons from at least one of the subset of scintillator arrays to a remainder of the subset of scintillator arrays. In some embodiments, the block structure can include an edge region. The at least one of the plurality of light guides in the edge region is coupled with a subset of scintillator arrays including a first scintillator array and a second scintillator array of the plurality of scintillator arrays in the edge region. In some embodiments, at least one of the plurality of photodetectors in the edge region comprises a prism structure configured to transfer photons from the first scintillator array to the second scintillator array.

In some embodiments, the block structure can include a corner region. In some embodiments, at least one of the plurality of light guides in the corner region is coupled with a subset of scintillator arrays including at least three of the plurality of scintillator arrays in the corner region. In some embodiments, at least one of the plurality of photodetectors in the corner region comprises a prism structure configured to transfer photons from a scintillator array from the subset of scintillator arrays on a corner to a remainder of the subset of scintillator arrays.

### BRIEF DESCRIPTION OF THE DRAWINGS

FIG. 1 depicts a perspective view of a device for detecting photons for tomography in accordance with an illustrative embodiment.

FIG. 2 depicts a top view of a light guide in accordance with an illustrative embodiment.

FIGS. 3A-3D depict a schematic of interleaved multiplexing scheme (iMUX) for the device for detecting photons for tomography.

FIG. 4 depicts a schematic of an example electric circuit to use the electric signal in accordance with an illustrative embodiment.

FIG. 5 depicts a perspective view of a system including a first block structure for tomographic imaging in accordance with an illustrative embodiment.

FIG. 6 depicts a top-perspective view of the first block structure in accordance with an illustrative embodiment.

FIG. 7 depicts a top-side view of the first block structure in accordance with an illustrative embodiment.

FIG. 8 depicts a top-perspective view of the first block structure in accordance with an illustrative embodiment.

FIG. 9 depicts a perspective view of a system including a second block structure for tomographic imaging in accordance with an illustrative embodiment.

FIG. 10 depicts a side-perspective view of the second block structure in accordance with an illustrative embodiment.

FIG. 11 depicts a top-perspective view of a dome light guide in accordance with an illustrative embodiment.

FIG. 12 depicts a bottom of the dome light guide in accordance with an illustrative embodiment.

FIG. 13 depicts a front perspective view of the dome light guide in accordance with an illustrative embodiment.

FIG. 14 depicts a top-perspective view of a scintillator crystal within the second block structure in accordance with an illustrative embodiment.

FIG. 15 depict a side-perspective view of the scintillator crystal within the second block structure in accordance with an illustrative embodiment.

FIG. 16 depicts a top-perspective view of one or more TOF DOI Prism-PET detectors in an interior region in accordance with an illustrative embodiment.

FIG. 17 depicts a top-perspective view of the one or more TOF DOI Prism-PET detectors in an edge region in accordance with an illustrative embodiment.

FIG. 18 depicts a top-perspective view of the one or more TOF DOI Prism-PET detectors in a corner region in accordance with an illustrative embodiment.

FIG. 19 depicts a flowchart of a method of arranging detector blocks for tomography in accordance with an illustrative embodiment.

FIG. 20 depicts three different scenarios in which gamma photons interact with Prism-PET module at different DOI.

FIG. 21 depicts (a) Light leak due to the finite gap between crystal and SiPM, which is the summation of the thickness of the coupling glue and protective resin (grey arrow), and due to the crystal column extends beyond the pixel's active area (green arrow). (b) Light leak is minimized by the tapered design. (c) Zoom-in schematic and (d) fabricated cross-sectional views of a 4-to-1 coupled Prism-PET detector module that crystals start tapering at 5 mm from the crystal-readout interface.

FIG. 22 depicts schematics of experimental setups: (a) DOI measurement, (b) Timing measurement.

FIG. 23 depicts Flood histograms and energy resolutions (uncorrected for saturation) for the (a) 1.4-mm cuboid, (b) 1.5-mm cuboid, and (c) 1.5-mm tapered Prism-PET module obtained with ²²Na uniform irradiation.

FIG. 24 depicts a flood map for the second block structure for tomographic imaging.

FIG. 25 depicts the energy resolutions for 1024 crystals of the second block structure for tomographic imaging.

FIG. 26 the DOI resolutions for 1024 crystals of the second block structure for tomographic imaging.

FIG. 27 depicts energy-weighted (w_{E}) DOI distributions, oTOF-weighted (w_{oTOF}) DOI distributions, and correlation between w_{E} and w_{TOF} for (a) 1.4-mm cuboid, (b) 1.5-mm cuboid, and (c) tapered Prism-PET detector module at five depths (2, 6, 10, 14, and 18 mm).

FIG. 28 depicts DOI distributions with energy-weighted (w_{E}) and three oTOF-weighted (w_{TOF}) methods for center, edge, and corner crystals in the tapered Prism-PET module at five depths (2, 6, 10, 14, and 18 mm).

FIG. 29 depicts DOI resolutions as a function of ratio r.

FIG. 30 depicts a histogram for energy based DOI (DOI_{E})

FIG. 31 depicts a histogram of oTOF-based DOI (DOI_{oTOF})

FIG. 32 depicts a timing spectra of the 1.4-mm cuboid (black triangle), 1.5-mm cuboid (blue square), and tapered (red circle) Prism-PET module using time offset correction with 3D channels including LSTS correction and LOR-based fine-tuning.

FIG. 33 depicts Axial sensitivity profiles of 10-ring Prism-PET brain scanner with 1.4-mm cuboid (green), 1.5-mm cuboid (red), and tapered (blue) modules.

### DETAILED DESCRIPTION

Following below are more detailed descriptions of various concepts related to, and embodiments of, systems, devices, apparatuses, and methods for detecting photons for tomography. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes systems, devices, and methods for detecting photons for tomography.

Section B describes depth-encoding using optical photon TOF in Prism-PET detector with tapered crystals.

### A. Systems, Devices, and Methods for Detecting Photons for Tomography

The present disclosure is directed to a detector block for a tomography scanner. The disclosed detector block can include scintillator crystals with a taper. Such scintillator crystals can allow for a reduction in photon leakage and improve energy detection by a photodetector.

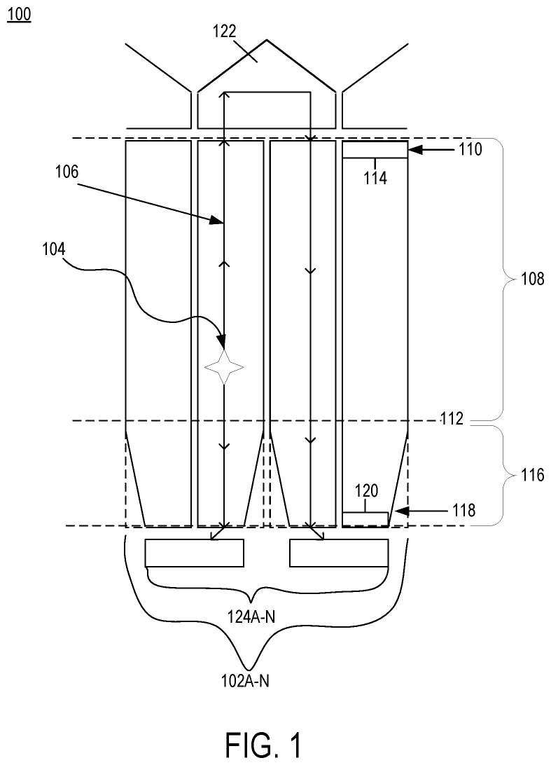

Referring now to FIG. 1, depicted is perspective view of a device 100 for a tapered Time-of-flight (TOF) depth of interaction (DOI) Prism-positron emission tomography (PET) detector 100 (referred to as “device 100” herein). In an overview, the device 100 can include a plurality of scintillator arrays 102A-N (referred to as “scintillator arrays 102” or a “scintillator array 102”), at least one of ionization radiation 104, at least one photon 106, at least one first region 108, at least one first end 110, at least one intermedial region 112, at least one first cross-sectional dimension 114, at least one second region 116, at least one second end 118, at least one second cross-sectional dimension 120, a plurality of light guides 122A-N (referred to as “light guides 122” or a “light guide 122”), and a plurality of photodetectors 124A-N (referred to as “photodetectors 124” or a “photodetector 124”), among others. The device 100 may correspond to the structure or apparatus as detailed herein Section B.

The device 100 can be an imaging device utilized in tomography (e.g., PET) for high resolution and detection of gamma rays emitted from radiotracers in biological tissues (e.g., brain). The device 100 can be constructed using a plurality of scintillation crystals (e.g., scintillator arrays 102) coupled to the light guide 122 and the photodetectors 124. The plurality of scintillation crystals can be a lutetium oxyorthosilicate (LSO) crystal, cerium-doped lutetium-yttrium oxyorthosilicate (LYSO) crystal, sodium iodide (NaI) crystal, alkali halide crystal, inorganic non-alkali crystal, bismuth germanate (BGO) crystal, or cesium iodide (CsI), among others. The device 100 can convert gamma-rays (e.g., ionization radiation 104) into visible light (e.g., photons 106). From here the device 100 can transmit the photons 106 to the light guide 122 and to the photodetector 124 for generating or reconstructing tomographic imaging.

The device 100 can include the plurality of scintillator arrays 102. The scintillator arrays 102 can be contained within the device 100. The scintillator arrays 102 can have a shape of a rectangular prism casing with a trapezoidal, rectangular, or polygonal base in the depicted example. A height of the scintillator array 102 can be greater than the width and the length of the scintillator array 104. For example, the scintillator array 102 can have a height of 5-20 mm and a maximum length and width of 1.0-3.5 mm. In some examples, the width of the scintillator array 102 can be greater than the length. In some examples, the length of the scintillator array 102 can be greater than the width. The scintillator array 102 can be formed to include a casing within a polygonal base, such as a triangle, a rectangle, a square, a pentagon, or a hexagon, for example.

The scintillator array 102 of the device 100 can a plurality of scintillator crystals to formulate the scintillator array 102. For example, the scintillator array 102 can include a 16×16 arrangement of scintillator crystals. The scintillator array 102 can be fabricated by a plurality of materials such LSO or LYSO to determine light output, energy resolution, decay time, among others. For example, if the scintillator array 102 is fabricated using LSO, each scintillator crystal can include a higher light yield and a faster decay time. The LSO material of the scintillator array 102 can have a higher resistance to radiation damage, thus improving the longevity of scintillator array 102. Furthermore, the LSO material can allow the scintillator array 102 to acquire high spatial and temporal resolution in TOF PET imaging.

Each scintillator crystal 102 in the scintillator array 102 can be configured to receive, obtain, or otherwise collect ionizing radiation 104. The scintillator crystal 102 can receive the ionizing radiation 104 within the first region 108, the intermedial section 112, and the second region 116. For instance, the scintillator crystal 102 can receive the ionizing radiation 104 within the first region 108. In another instance, the scintillator crystal 102 can receive the ionizing radiation 104 in the intermedial section 112. In yet another instance, the scintillator crystal 102 can receive the ionizing radiation 104 in the second region 116. The scintillator crystal 102 can receive the ionizing radiation 104 through scintillation. In some instances, the ionizing radiation 104 can disperse over an area of the scintillator crystal 102 in the depicted example.

The ionizing radiation 104 can be a form of energy that can isolate, liberate, or otherwise release electrons from atoms. By releasing the electrons, the ionizing radiation 104 can create ions. The ionizing radiation 104 can originate from a plurality of sources such as radioactive decay, nuclear reactions, electromagnetic phenomena (e.g., X-rays, Gamma rays, alpha particles, beta particles). For example, the electromagnetic phenomena can transmit gamma rays to the scintillator crystal 102 as the ionizing radiation 104, as part of positron emission tomography (PET) or single photon emission computer tomography (SPECT). When the ionizing radiation 104 collides with the atoms, the ionizing radiation 104 can provide or transfer energy to the released electrons. The transferred to the electrons can result in an emission of photons 106 within the scintillator crystal 102. In some instances, the emission of photons 106 can occur in fluorescence or luminescence. For example, during fluorescence, the scintillator crystal 102 can produce photons 106 of a plurality of characteristic energies based on the atoms within the scintillator crystal 102.

The scintillator crystal 102 can be configured to emit the photons 106 in response to the reception of the ionizing radiation 104. The photons 106 of the scintillator crystal 102 can be referred to ask scintillation light. The photons 106 can be in the visible range or the ultraviolet range. For instance, the photons 106 can be within the ultraviolet range (e.g., 10 nanometers (nm) to 400 nm. In another instance, the photons 106 can be within the visible light range (e.g., 400 nm to 700 nm). In some instances, the photons 106 can be within at least one of the three categories of the UV spectrum. The photons 106 can include a plurality of colors (e.g., violet, blue, green, yellow, orange, and red) when the scintillator crystal 102 received the ionizing radiation 104.

The scintillator crystal 102 can emit, transmit, or generate the photon 106 from the first region 108, the intermedial section 112, or the second region 116 based on the received ionizing radiation 104. For example, when the scintillator crystal 102 receives the ionizing radiation 104 in the first region 108, the photon 106 can emit from the first region 108. In another instance, when the scintillator crystal 102 receives the ionizing radiation 104 in the second region 116, the photon 106 can emit from the second region 116. During the emission process, one or more photons 106 can move, proceed, or otherwise advance in a direction away from area of the ionizing radiation 104, as shown in the depicted example. The direction can be towards the light guide 122 or the photodetector 124. For instance, when the scintillator crystal 102 receives the ionizing radiation 104 at the first region, one or more photons 106 can proceed toward the light guide 122, whereas one or more photons 106 can proceed to the photodetector 124.

Each scintillator crystal 102 can include the first region 108, the intermedial section 112, and the second region 116 defined throughout the scintillator crystal 102. In the example depicted in FIG. 1, among others, the first region 108, the intermedial section 112, and the second region 116 can be defined along a latitudinal axis of the scintillator crystal 102. The first region 108, the intermedial section 112, and the second region 116 can be defined by an exterior surface of the scintillator crystal 102. The first region 108 can be on an upper portion of the latitudinal axis. The intermedial region 112 can be fined by the latitudinal axis and in between the first region 108 and the second region 116. The second region 116 can be on a lower portion of the latitudinal axis opposite the first region 108.

The first region 108 of the scintillator crystal 102 can be above the intermedial section 112 and opposite to the second region 116. The first region 108 can be coupled to or interface with the light guide 122. The first region 108 can be an entry point or exit point for the photons 106, during the emission process. The first region 108 can include dimensions and capabilities to optimize photon 106 collection and photon 106 transmission. In some instances, the first region 108 can be polished, formatted, or trimmed to minimize imperfections, improve photon 106 transmission, and reduce photon 106 scattering. In some instances, the scintillator crystal 102 can include a layer of coating or treatment to improve reflectivity and enhance photon 106 detection. The first region 108 can correspond to the first end 110 of the scintillator crystal 102.

The first end 110 of the scintillator crystal 102 can be the edge of the scintillator crystal 102 that interacts with and coupled to the light guide 122. The first end 110 can be a certain distance away from the intermedial section 112. For instance, the first end 110 can be greater than 8 mm away from the intermedial section 112. In another instance, the first end 110 can be less than 10 mm away from the intermedial section 112. The first end 110 can be configured to transmit photons 108 to the light guide 122 and receive photons 106 from the light guide 122. For instance, the photon 106 can pass through the first end 110 of a first scintillator crystal 102 to reach the light guide 122. From here the photon 106 can enter a second scintillator crystal 102 through the first end 110.

The intermedial section 112 of the scintillator crystal 102 can include the same material as the scintillator crystal 102. In some instances, the intermedial section 112 can be polished or trimmed similar to the first region 108 to optimize photon 106 movement and increase reflectivity. For example, the intermedial section 112 can be configured to maximize the movement speed of the photon 106 within the scintillator crystal 102. The intermedial section 112 can be defined by the latitudinal axis going across each scintillator crystal 102 in the scintillator array 102. The latitudinal axis can be defined by the midpoint of each scintillator crystal 102 located within the intermedial section 112. Using the latitudinal axis, intermedial section 112 can be equidistant from the first region 108 and the second region 116 or equidistance from the first end 110 and the second end 118. The intermedial section 112 can have a length and width similar to the length and width of the scintillator crystal 102.

The intermedial section 112 can include the first cross-section dimension 114 as defined by the length and width of the scintillator crystal 102. The first cross-section dimension 114 can define a path for the photon 106. For instance, the first cross-section dimension 114 can maintain a path for the photon 106 to travel through the light guide 122 and travel to the photodetector 124. The first cross-section dimension 114 can have a width of 1.0 mm-3.5 mm. The first cross-section dimension 114 can be the same as the dimension of the first end 110.

The first end 110 can have a cross-sectional dimension smaller than the first cross-section dimension first cross-section dimension 114 by the first end including a taper similar to the taper of the second end 118 described herein. The cross-sectional dimension of the first end 110 can allow the photons 106 to enter the scintillator crystal 102 with higher accuracy and less photon 106 leakage. For instance, the taper at the first end 110 can prevent or reduce the loss of the photons 106 transmitted from the light guide 122. In some instances, the taper at the first end 110 can be the same as taper at the second end 118 of the scintillator crystal 102 described herein.

The second region 116 of the scintillator crystal 102 can be below the intermedial section 112 and opposite to the first region 108. The second region 116 can be coupled to or interface with the photodetector 124. The second region 116 can be an exit point for the photons 106, during the emission process. The second region 116 can include dimensions and capabilities to optimize photon 106 transmission. In some instances, the second region 116 can be polished, formatted, or trimmed to minimize imperfections, improve photon 106 transmission, and reduce photon 106 scattering. In some instances, the scintillator crystal 102 can include a layer of coating or treatment to improve reflectivity and enhance photon 106 detection. The second region 116 can correspond to the second end 118 of the scintillator crystal 102.

The second end 118 of the scintillator crystal 102 can be the edge of the scintillator crystal 102 that interacts with and is coupled to the photodetectors 124. The second end 118 can be a certain distance away from the intermedial section 112. For instance, the second end 118 can be greater than 2-8 mm away from the intermedial section 112. In another instance, the second end 118 can be less than 5-10 mm away from the intermedial section 112. The second end 118 can be configured to transmit photons 1108 to the photodetectors 125. For instance, the photon 106 can pass through the second end 118 of a first scintillator crystal 102 to reach the photodetectors 124.

The second end 118 can include a second cross-section dimension 120 as defined by the scintillator crystal 102. The second cross-section dimension 120 can define a path for the photon 106 to reach the photodetector 124. For instance, the second cross-section dimension 120 can maintain a path for the photon 106 to travel to the photodetectors 124. In some instances, the second cross-section dimension 120 can include a dimension of 1.2 mm-1.5 mm which is the same as the first end 110 and the intermedial section 112. In some instances, the second cross-section dimension 120 can be smaller than the first cross-section dimension 114. For instance, the first cross-section dimension 114 can have a width of 1.4 mm-1.5 mm whereas the second cross-section dimension 120 can have a width of 1.2 mm-1.5 mm.

The second cross-section dimension 120 can be formed by trimming away 0.1 mm-0.3 mm from the width of the scintillator crystal 102 to form a taper in the scintillator crystal 102. During the trimming process, the taper of the scintillator crystal 102 can start approximately 0.3 mm-0.5 mm away from the second end 118. For instance, the taper of the scintillator crystal 102 can have a height of 0.2 mm-0.5 mm. The taper of the scintillator crystal 102 can be at least one of linear, polynomial, logarithmic, exponential, quadratic, trigonometric, among others. The taper of the scintillator crystal 102 can provide a plurality of benefits for mitigating photon 106 leak in the scintillator array 102.

The taper can be a linear cut of the scintillator array 102, defined by a straight line starting from at most 0.5 mm away from the second end 118. The taper of the scintillator crystal 102 can define the path for the photons 106 to travel into the photodetector 124 by guiding he photons 106 into the exit point. For instances, as the photons 106 travel down the scintillator crystal 102, the taper can direct photons 106, that would otherwise leak from the scintillator array 102, into the exit point. In his manner, the taper can provide the photometric 124 with more photons 106 than previously achieved. Furthermore, the taper can minimize the loss of photons 106 within the scintillator crystal scintillator array 102, by using the linear cut. In some instances, a polynomial cut (e.g., quadratic, cubic, quartic) can define the path for the photons 106 in a similar manner as the linear cut. The polynomial cut can correspond to the dimensions of the scintillator crystal 102 and the area of reception for the ionizing radiation 104. In some instances, for larger scintillator crystal scintillator array 102 with a more difficult path for the photos 108, the polynomial cut can define the path and minimize the photon 106 loss.

The taper of the scintillator crystal 102 can include an angle of depression formed between the start point of the trim and the second end 118. The angle of depression can vary based on a change in the start point of the trim and in the second cross-section dimension 120. For instance, if the trim started at a height of 0.3 mm instead of 0.5 mm, the angle of depression can increase and reduce the quality of the path for the photons 106. Despite more photon 106 loss at 0.3 mm, the photon 106 loss may be less than if the taper did not exist. In another instance, the second cross-section dimension 120 can increase to 1.4 mm, thereby increasing the angle of depression. The increased angle of depression may minimize the photon 106 loss, but less than the photon 106 loss at the second cross-section dimension 120 of 1.2 mm.

The taper of a first scintillator crystal 102 can differ from the taper of a second scintillator crystal 102. For instance, the taper of the first scintillator crystal 102 can occur on the left edge of the first scintillator crystal 102, whereas the taper of the second scintillator crystal 102 can occur on the right edge of the second scintillator crystal 102. In some instances, the second region 116 of the scintillator crystal 102 can have a new cross-section dimension tapering from the first cross-section dimension 114 to the new cross section dimension to minimize leakage of photons 106 to a second photodetector neighboring the photodetector. The taper can define an angle of departure for the photon 106 exiting the scintillator crystal scintillator array 102, as shown in the depicted example. Using the taper, more photons 106 can be focused through the angle of departure at the exit point to allow for increased energy at the photodetector 124. The result of the benefits of the taper are described in Section B.

The device 100 can include a light guide 122 coupled to the first end 110 of one or more scintillator crystals 102 in the scintillator array 102, as shown in the depicted example. For instance, one light guide 122 can be coupled to two scintillator crystals 102. In another instance, the light guide 122 can be coupled to four scintillator crystals 102. In yet another instance, the light guide 122 can be coupled to three scintillator crystals 102. The light guide 122 can have a base to match the dimension of one or more first cross-section dimensions 114 depending on the one or more scintillator crystals 102 coupled to the light guide 122. The light guide 122 can be a prism structure (e.g., rectangular, cub, triangular, pentagonal, hexagonal, etc.) to facilitate the transfer of photons 106 between the scintillator array 102.

The light guide 122 positioned atop scintillator crystals 102 can be a configured to transfer the photons 106 from a first scintillator crystal 102 to a second scintillator crystal 102. For instance, the light guide 122 can transfer photons from a first scintillator crystal 102A to a second scintillator crystal 102B. The light guide 122 can allow for efficient capture and transmission of the photons 106 and light signals generated within the scintillator material. The light guides 122 can be made of acrylics, optical glasses, polymers, among others. The material can be chosen based on desired transparency and light-conducting properties. The design of the light guide 122 can consider shape, dimensions, surface treatments, among others, to minimize light loss and maximize signal fidelity. Furthermore, an interface between the scintillator crystal 102 and the light guide 122 can allow for optimal coupling, reducing reflection and scattering losses.

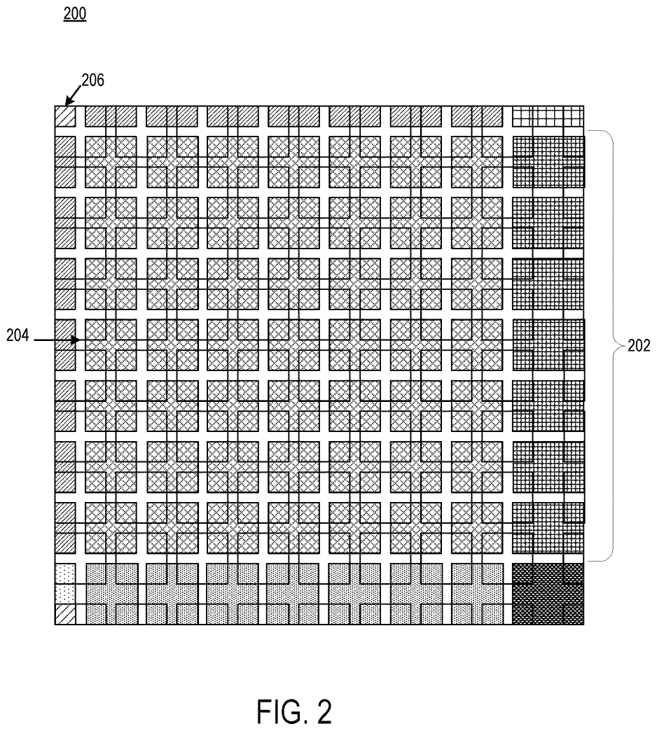

Referring now to FIG. 2, depicted is a mapping 200 of the different types of light guide 122. The light guides 122 can include at least one edge light guide 202, at least one center light guide 204, and at least one corner light guide 206. Each of the light guides 122 in the mapping 200 correspond to a plurality of regions of a block structure described herein. For instance, the at least one edge light guide 202 can correspond to an edge region of the block structure. In another instance, the at least one center light guide 204 can correspond to a center region of the block structure. In yet another instance, the at least one corner light guide 206 can correspond to a corner region of the block structure.

Referring again to FIG. 1, the device 100 can include one or more photodetectors 124 coupled to the second end 118 of the one or more scintillator crystals 102 of the scintillator array 102. For instance, the photodetectors 124 can be coupled to at least two of the scintillator crystals 102. In another instance, the photodetector 124 can be coupled to four scintillator crystals 102. The photodetectors 124 can have a dimension greater than or equal to the second cross-section dimension 120. The photodetectors 124 can include a cross-section dimension corresponding to a cross-section dimension defined by the second cross-section dimension 120 of at least two scintillator crystal 102. For instance, two scintillator crystal 102 can each include a second cross-section dimension 120. The two second cross-section dimensions 120 can define the cross-section dimension of two photodetectors 124 as shown in FIG. 1.

The photodetector 124 can transform, convert, or provide the received photons 106 from the scintillator array 102 into an electrical signal for tomography imaging. Photodetectors 124, under scintillator crystals 102, can be fabricated from various materials, including photomultiplier tubes (PMTs), silicon photomultipliers (SiPMs), avalanche photodiodes (APDs), among others. By the scintillator crystal 102 including the taper, the photo detector 124 may provide more accurate readings of the photons 106 to an electrical circuit.

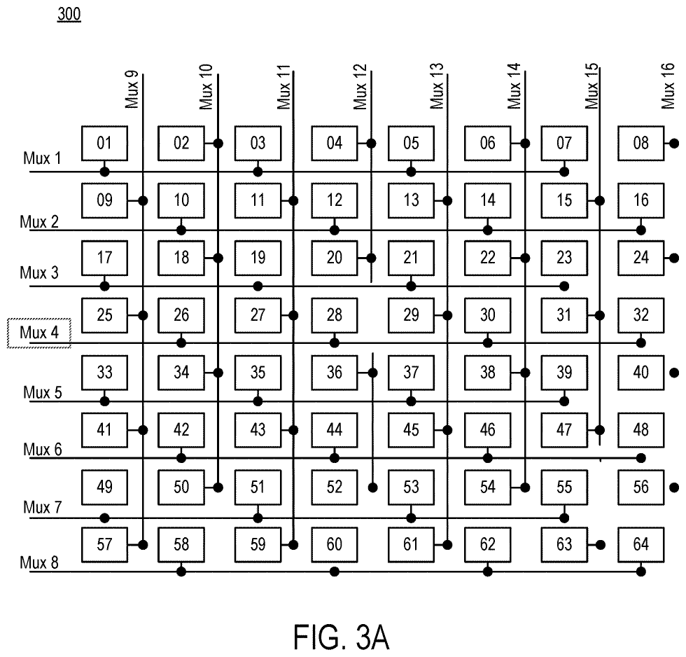

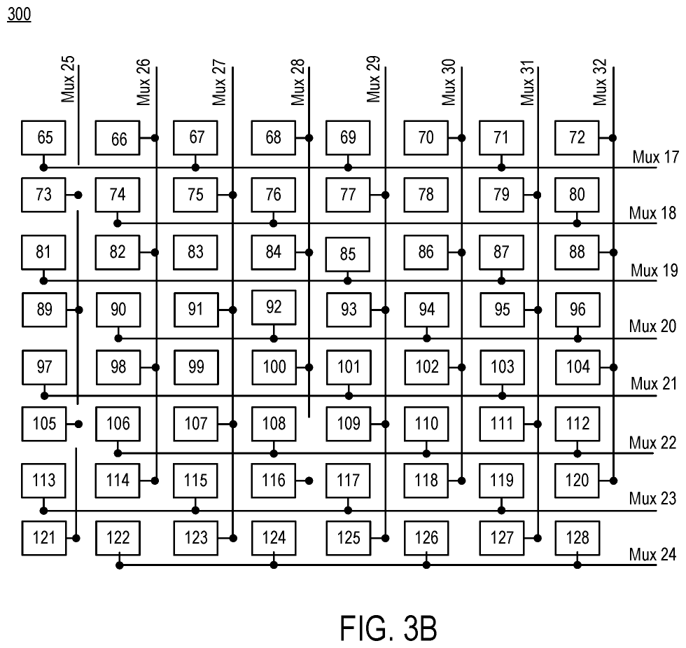

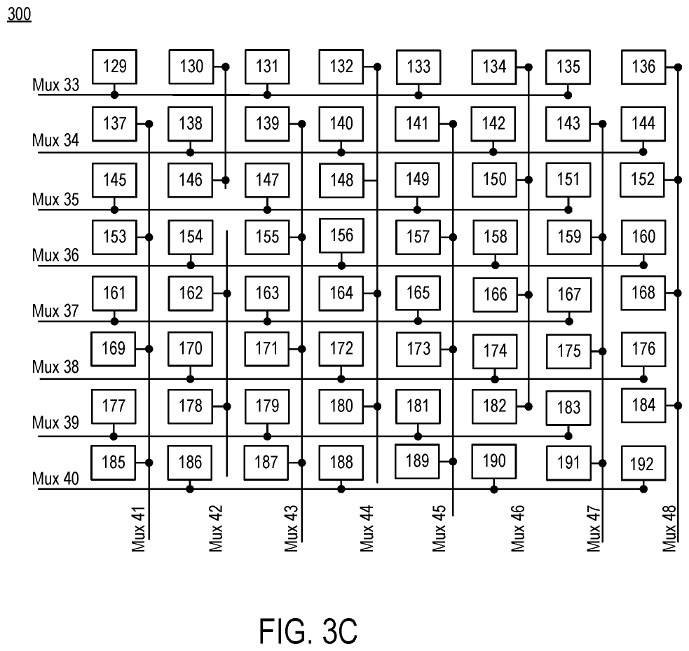

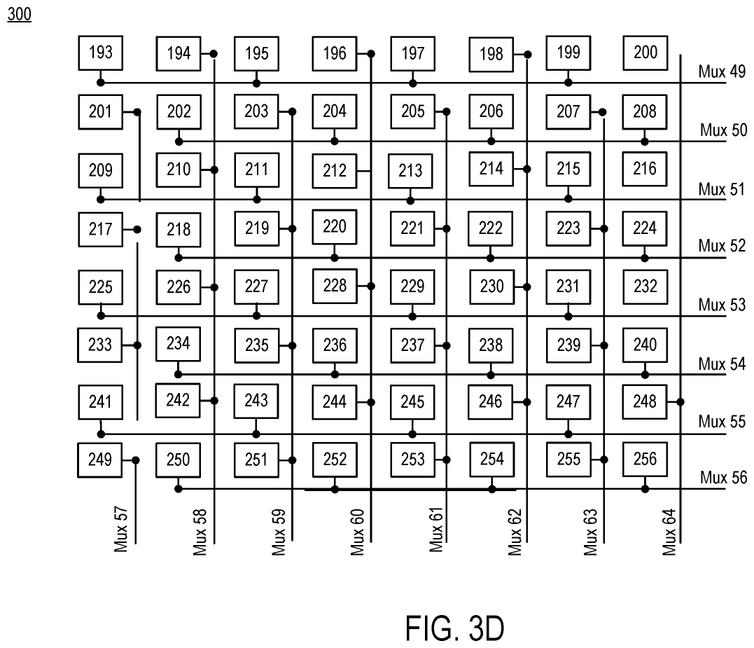

Referring now to FIGS. 3A-D, depicts a schematic 300 (generally referred to as “electric circuit 300”) of interleaved multiplexing scheme (iMUX) for the device 100. The pixels 302A-N (generally referred to as a “pixel 302” or “pixels 302”) can be SiPM pixels 302. Each pixel 302 can be connecting using a plurality of wires where signals from every other 4 SiPM pixels 302 across rows and columns are shorted together to connect to the same ASIC channel. The iMux scheme, due to its unique connecting pattern, does not accumulate all the pixel energy from a single row or column in a given multiplexed readout channel, which can lead to a loss of spatial information when using the conventional technique in gamma-ray event reconstruction of photodetectors, such as center of gravity (CoG), resulting in a distorted flood histogram that fails to accomplish crystal identification. Therefore, aspects of the technical solution use a “weighted compensation (WC)” approach (as shown in the below formula) to address this issue, which takes advantage of the deterministic energy pattern on the detector modules and the interleaved connecting scheme to appropriately compensate for the missing energy information in each multiplexed readout channel.

$$\hat{m} = m + β \cdot {m}_{⊥} ,$$

where \hat{m} is compensated iMux ASIC channel value, m is measured iMux ASIC channel value, β is compensation factor, and m_{⊥} is iMux ASIC value of the perpendicular row/column since each multiplexed channel is missing the energy information of 4 SiPMs that are contained in the other 4 channels perpendicular to it.

$$X = \frac{{\sum }_{i = 1}^{8} {m}_{{x}_{i}}^{α} {x}_{i}}{{\sum }_{i = 1}^{8} {m}_{{x}_{i}}}$$

$$Y = \frac{{\sum }_{j = 1}^{8} {m}_{{y}_{i}}^{α} {y}_{i}}{{\sum }_{j = 1}^{8} {m}_{{y}_{j}}} ,$$

where m is energy value of each readout channel; α is optimization parameter of raise-to-the-power (RTP) algorithm, which aims to achieve the optimal average peak-to-valley ratio (PVR) and the best peak separation at the edges of flood images; x, y are coordinates of the pixel in the SiPM array; X, Y are coordinates in the flood map.

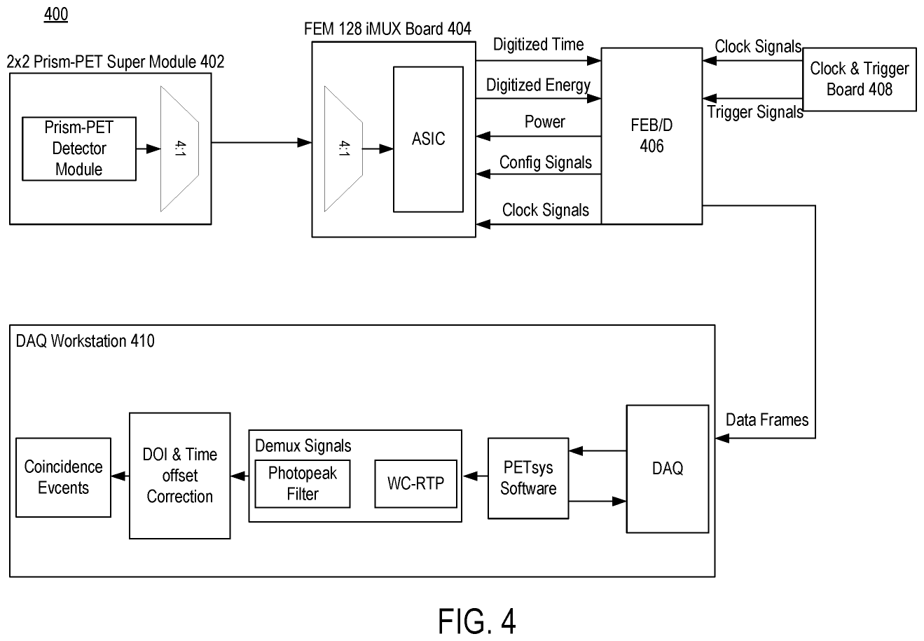

FIG. 4, depicts an example block diagram 400 of the electric circuit 300 to use the electrical signal for tomography imaging. The device can include a 2×2 Prism-PET Super Module 402 (described as a block structure herein) that includes a 32×32 array of 1.5×1.5×20 mm3 scintillator crystals, which can be tapered down to 1.2×1.2 mm2 at the crystal-readout interface. The tapered crystal array 102 can be coupled 4-to-1 to an 8×8 array of 3×3 mm2 SiPM pixels 302 on the tapered side and to a dome or prismatoid light guide 122 array on the opposite side. The block diagram 400 can execute data acquisition using FEM boards 404 each including multiplexing electronics and 2 TOFPET2 application specific integrated circuit (ASIC) chips reading 128 channels, FEB/D v2 readout boards 406, and a Clock&Trigger module 408. The coincidence data acquired can be processed with the WC-RTP demultiplexed algorithm, photopeak contour filter, and DOI& time offset correction within the DAQ workstation 410. The block diagram 400 can be configured to perform TOF or DOI using the electrical signal for tomography imaging. For instance, using the electrical signal as an input, the block diagram 400 can perform TOF for tomography imaging.

The TOF can enhance the images of the PET scanners. The TOF can measure, indicate, or otherwise identify the time it takes for the photon 106 to travel through the emission point to the photodetectors 124. For instance, the TOF can identify the time in which the photon 106 travels across the scintillator crystal 102 into the photodetector 124. By analyzing the TOF, a computing device can determine a level of improvement between the scintillator array 102 without the taper and the scintillator array 102 with the taper.

The DOI for the PET detectors can determine, identify, or calculate a distance within the scintillator crystal 102 where the ionizing radiation 104 can impact. For example, the DOI can correspond to a depth of 0.1 mm-0.5 mm for the received ionizing radiation 104. The DOI can enhance the spatial resolution of the tomographic images by determining the depth of the ionizing radiation 104. Using the DOI, the computing system can generate a reduction in photons 106 loss based on the taper enhancing the strength of the photons 106 at the photodetectors 124.

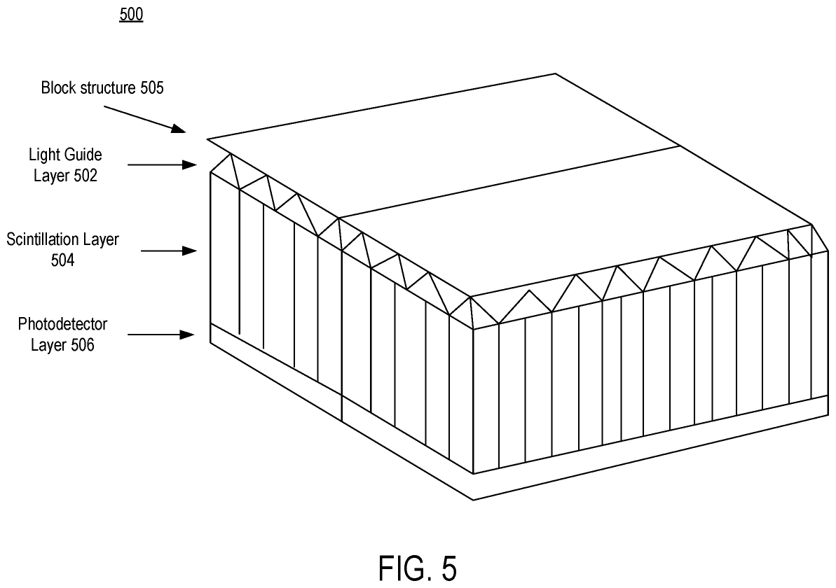

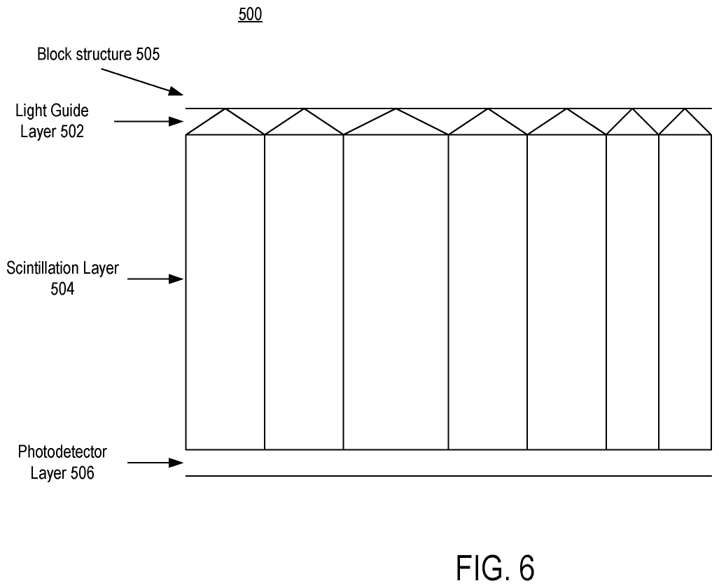

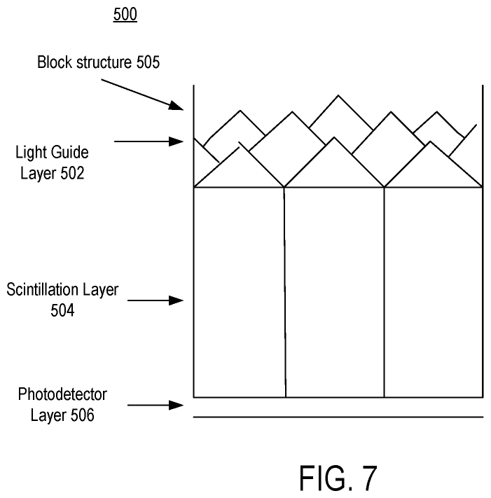

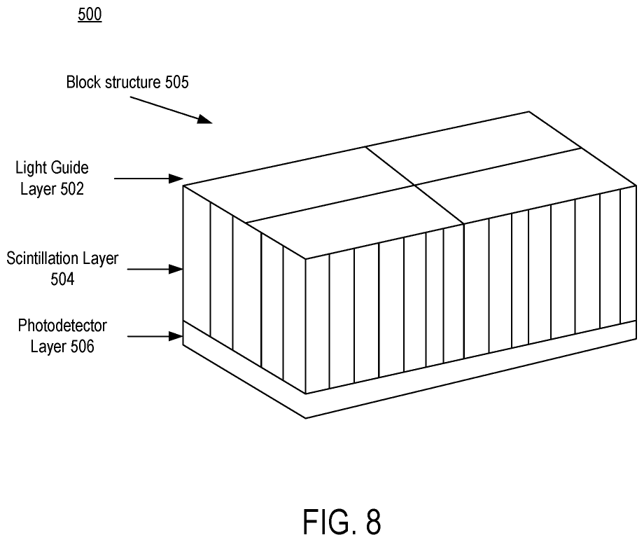

Referring now to FIGS. 5-8, depicted are multiple perspective views of a system 500 including a block structure 505 for tomographic imaging. The block structure 505 can include the plurality of scintillator arrays 102 described above. Using the plurality of scintillator arrays 102, the block structure 505 can be a 2×2 module that include a 32×32 scintillator array 102. The block structure 505 can include multiple configurations of the scintillator arrays 102, light guides 122, and photodetectors 124 spread across three layers. The layers can include a light guide layer, a scintillator layer, and a photodetector layer. The light guide layer 502 of the block structure 200 can include the light guides 122 directly coupled the to the scintillation layer 504. The scintillator layer 504 can include the plurality of scintillator arrays scintillator array 102, directly coupled to the light guide layer 502 and the photodetector layer 506. The photodetector layer 506 can include the photodetectors 124 directly coupled to the scintillation layer 504.

The light guides 122 can be arranged along the light guide layer 504 as shown in FIGS. 5-8. The light guides 122 can be arranged according to the scintillator array 102. The scintillator array 102 can be arranged along the scintillation layer 504 a number of scintillator crystal 102 per column and row. FIGS. 5-8 depicts the block structure 505 with a 16×16 scintillator array 102. As such, the light guides 122 can be arranged in a 16×16 array to match the scintillator array 102. In some instances, the light guides 122 can be arranged in an array less than the scintillator array 102. For instance, if two scintillator crystals 102 couple to one light guide 122, the light guide 122 array can be 16×8 despite the scintillator crystals 102 arranged in a 16×16 scintillator array 102. Thus, the light guides 122 can couple to at least two scintillator crystals 102 in the scintillator array 102. Similar to the light guides 122, the photodetectors 124 can be arranged in the photodetector layer 506 in an array less than the scintillator array 102 and couple to at least two scintillator crystals 102 of the scintillator array 102.

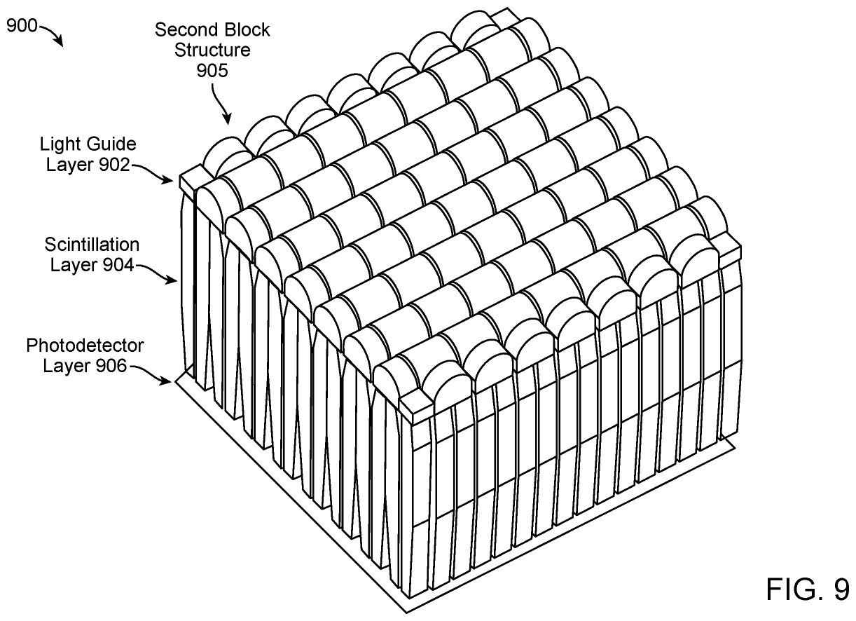

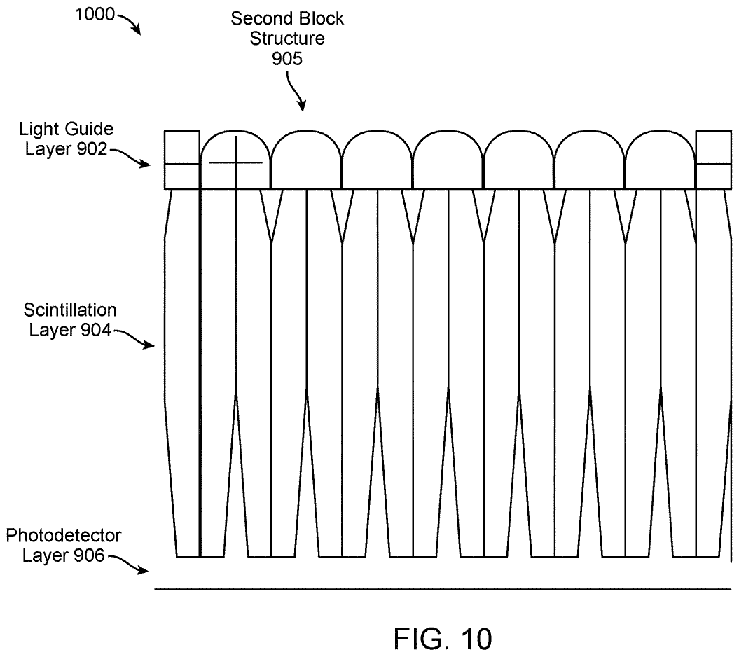

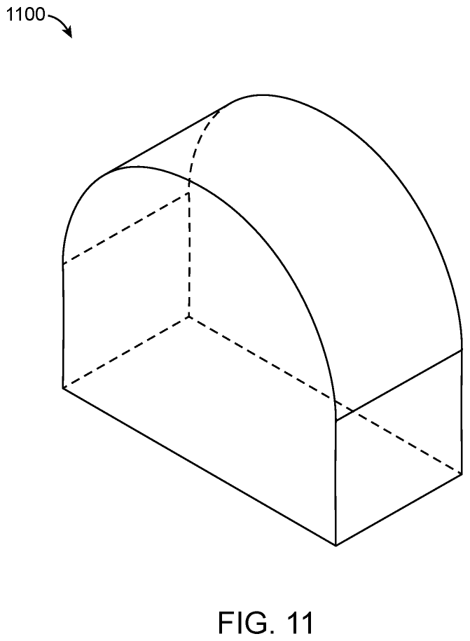

Referring now to FIGS. 9-10, depicted multiple perspective views of a system 900 including a second block structure 905 (referred to as “HexaDome 905” for tomographic imaging.) The second block structure 905 can allow for high resolution, high sensitivity, and cost-effective SPECT and PET scanner. The second block structure 905 can be similar to the block structure 505. The second block structure 905 can include multiple configurations of the scintillator arrays 102, light guides 122, and photodetectors 124 spread across the three layers. The layers can include a light guide layer 902, a scintillator layer 904, and a photodetector layer 906. The light guide layer 902 can include a plurality of dome shaped (e.g., Hemisphere, Ellipsoid dome, Ovoid Dome, Quarter Sphere, among others) light guides 122 directly coupled to the scintillator layer 904. The scintillator layer 904 can include a plurality of hexagon shaped (e.g., hexagonal prism, regular hexagon, hexagonal bipyramid, among others) scintillator arrays 102 directly coupled to the photodetector layer 906 and the light guide layer 902. The photodetector layer 906 can include the photodetectors 124 directly coupled to the scintillator layer 904.

The second block structure 905 can be a polygonal shape, such as a rectangular prism, a cube, a cuboid, a parallelepipeds, and the like. The polygonal shape can be similar to the block structure 505, allowing the second block structure 905 a similar arrangement of scintillator arrays 102. For example, the block structure 505 can include a 16×16 array of insulators, therefore, the second block structure 905 can include a 16×16 array of scintillators. The second block structure 905 can include dimensions different from the dimensions of the block structure 505. For example, the height of the block structure 505 can be 5 mm-20 mm, whereas the height of the second block structure 905 can be 7 mm-10 mm.

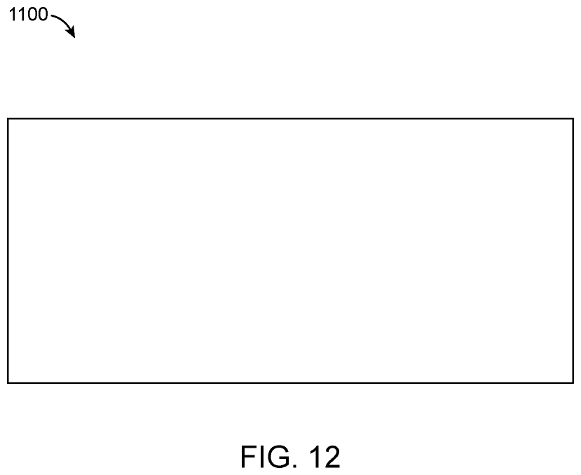

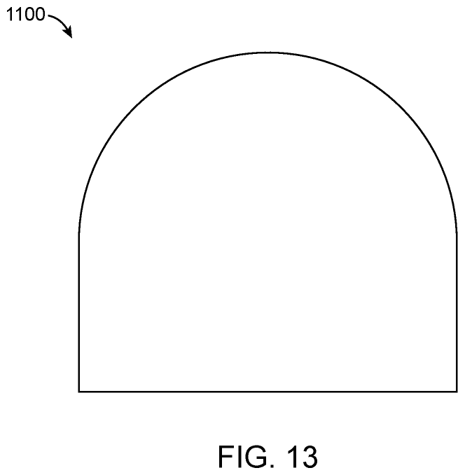

Referring now to FIGS. 9-13, depicted are multiple perspective views of a dome light guide 1100. The light guide layer 902 can include a plurality of dome light guides 1100 coupled to the scintillator layer 904. The dome light guide 1100 can be arranged above one or more scintillator crystals 102 in the plurality of scintillator arrays 102. For example, one dome light guides 1100 can be arranged above two scintillator crystals 102. In another example, two dome light guides 1100 can be arranged above three scintillator crystals 102. The dome light guides 1100 can be arranged in a configuration similar to the plurality of light guides 122. Dimensions of the dome light guides 1100 can vary in different configurations for the second block structure 905. For example, in a first second block structure 905 configuration, the length of a first dome light guide 1100 can be 1.0 mm-1.2 mm. However, in a second block structure 905 configuration, the length of a second dome light guide 1100 can be 0.9 mm-1.1 mm. The curvature of the dome light guide 1100 can distribute photons 106 evenly across the surface of the dome light guide 1100 to reduce the photon loss in the system 500. The dome light guide 1100 can include materials similar to the light guides 122.

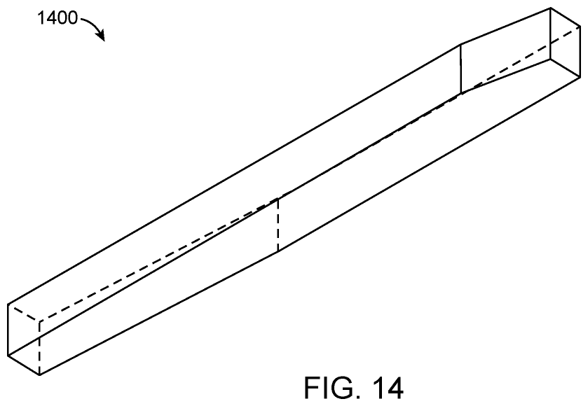

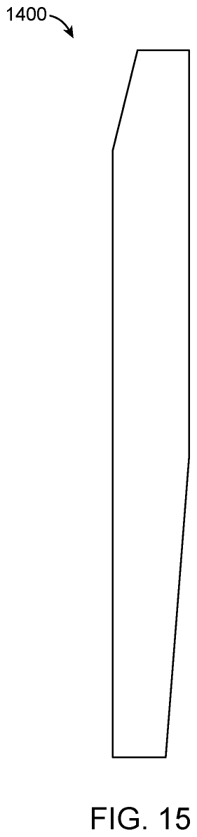

Referring now to FIGS. 9-15, depicted are multiple perspectives of the scintillator crystals 1400 (referred to as “hexagon-shaped crystals 1400” herein). The scintillator layer 904 can include a plurality of hexagon shaped crystals 1400 coupled to the light guide layer 902 and the photodetector layer 906. The hexagon shaped crystals 1400 can be sandwiched between the plurality of dome light guides 1100 and the photodetectors 124. The hexagon shaped crystals 1300 can be arranged similar to the scintillator crystals 102 within the scintillator array 102. Dimensions for the hexagon shaped crystals 1400 can vary in different configurations for the second block structure 905. For example, in a first second block structure 905 configuration, the length of a first plurality of hexagon shaped crystals 1400 can be 6.0 mm-7.0 mm. However, in a second block structure 905 configuration, the length of a second plurality of hexagon shaped crystals 1400 can be 7.0 mm-8.0 mm.

Each of the hexagon shaped crystals 1400 can include the taper on the first region 108 and the second region 116 similar to some scintillator crystals 102. The dimensions of the hexagon shaped crystals 1400 can vary from the scintillator crystals 102 based on the depth of the taper. The hexagon shaped crystals 1400 can include a taper with length greater than the taper of the scintillator crystals 102. For example, the length of the taper of the hexagon shaped crystals 1400 can be 1.00 mm-3.00 mm, whereas the length of the taper of the scintillator crystals can be 0.3 mm-0.5 mm. The longer taper of the hexagon shaped crystals 1400 can reduce photon loss more efficiently than the taper of the scintillator crystal 102 by allowing my photons 106 to interact with the exit point of the hexagon shaped crystals 1400.

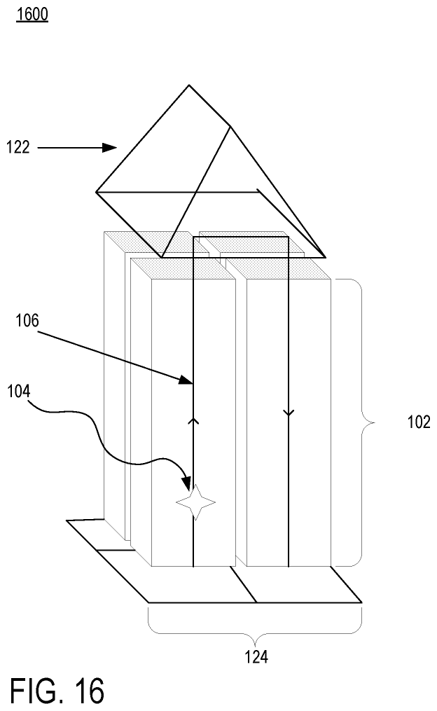

Referring now to FIG. 16, depicted is a top-perspective view of one or more TOF DOI Prism-PET devices 100 in an interior region 1600. The interior region 1600 can be located in any location of the block structure 505 that is not adjacent to an edge or a corner of the block structure 505. As shown in FIG. 16, a subset of the scintillator array 102 can include at least four scintillator crystals 102 and be coupled to at least one of the light guides 122. Furthermore, the photodetectors 124 can include a prism structure (e.g., rectangular, cubic) configured to transfer photons 106 from at least one scintillator crystal 102 in the subset (e.g., the other three) of the scintillator array 102 to the remainder of scintillator crystals 102 in the subset.

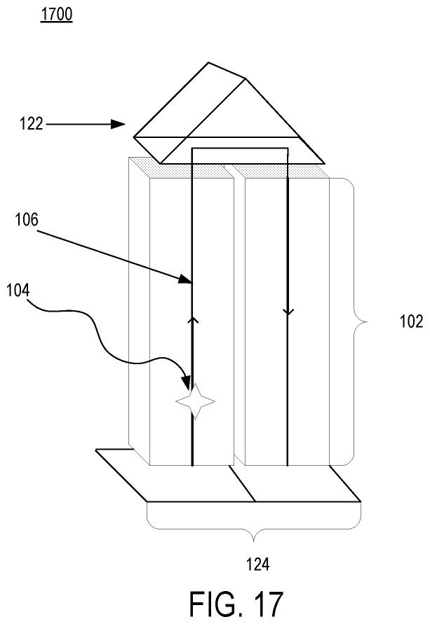

Referring now to FIG. 17, among others, depicted is a top-perspective view of one or more TOF DOI Prism-PET devices 100 in an edge region 1700. The edge region 1700 can be located along an outer portion of the block structure 505. The scintillator crystal 102 along the edge region 1700 can be adjacent to at least one scintillator crystal 102 as shown in FIG. 17. A subset of the scintillator array 102 can include at least two scintillator crystals 102 and be coupled to at least one light guide 122. Furthermore, the photodetectors 124 can include a prism structure (e.g., rectangular, cubic) configured to transfer photons 106 from at least one scintillator crystal 102 in the subset of the scintillator array 102 to the remainder of scintillator crystals 102 in the subset.

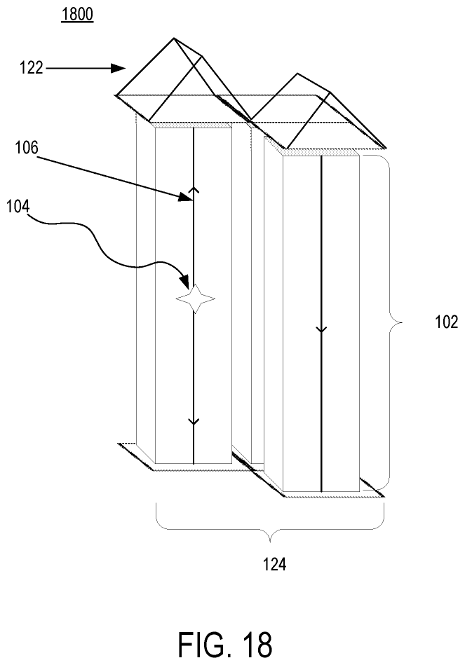

Referring now to FIG. 18, depicted is a top-perspective view of one or more TOF DOI Prism-PET devices 100 in a corner region 1800. The corner region 1800 can be located at an intersection of two adjacent sides of the block structure 505. The scintillator crystal 102 the corner region 1700 can be adjected to at least two scintillator crystals 102 as shown in FIG. 18. A subset of the scintillator array 102 can include at least three scintillator crystals 102 and be coupled to at least one light guide 122. Furthermore, the photodetectors 124 can include a prism structure (e.g., rectangular, cubic) configured to transfer photons 106 from at least one scintillator crystal 102 on the corner region 1800 in the subset of the scintillator array 102 to the remainder of scintillator crystals 102 in the subset.

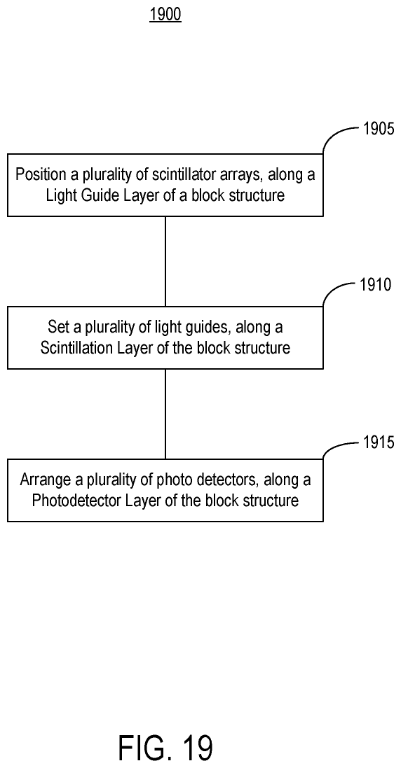

Referring now to FIG. 19, among others, depicts a flow diagram of method 1900 to form the block structure of TOF DOI Prism-PET detectors. The method 1900 may include positioning, along a layer of the block structure, a plurality of scintillator arrays (1905). To position the plurality of scintillator arrays, each scintillator crystal within the scintillator array can be placed equidistant from another scintillator crystal. Prior to positioning the plurality of scintillator array, each scintillator crystal can be trimmed on a region coupled to the photodetector to add a taper. Once trimmed, the scintillator crystal can be positioned according to a dimension of the tampered scintillator crystal and a photodetector. each respective scintillator array of the plurality of scintillator arrays can include a first region and second region. The first region can correspond to a first end to an intermedial section of the respective scintillator array, the intermedial section having a first cross-sectional dimension. The second region can correspond to the intermedial section to a second end of the respective scintillator array, the second end having a second cross-sectional dimension smaller than the first cross-sectional dimension

The method 1900 may include setting or positioning, along another layer of the block structure, a plurality of light guides (1910). The light guides can be set above the plurality of scintillator arrays. Once the plurality of scintillators arrays is positioned, the light guides can be set based on the location of each scintillator array within the block structure. For instance, an edge light guide can be set on a scintillator array in the edge region. In another instance, a center light guide can be set on a scintillator array in the center region.

The method 1900 may include arranging, along yet another layer of the block structure, a plurality of photodetectors. The plurality of photodetectors can be arranged based on the position of the plurality of the scintillator arrays. For instance, at last two photodetectors can be arranged under at least two scintillator crystals with the plurality of the scintillator arrays. The cross dimension of the scintillator crystals can match with the cross dimension of the photodetectors. In this manner, the photons can transmit to the photodetector thereby, reducing the amount of photon leakage.

### B. Depth-Encoding Using Optical Photon TOF In Prism-PET Detector With Tapered Crystal

High-resolution brain positron emission tomography (PET) scanner is emerging as a significant and transformative non-invasive neuroimaging tool to advance neuroscience research as well as improve diagnosis and treatment in neurology and psychiatry. Time-of-flight (TOF) and depth-of-interaction (DOI) information provide markedly higher PET imaging performance by increasing image signal-to-noise ratio and mitigating spatial resolution degradation due to parallax error, respectively. PET detector modules that utilize light sharing can inherently carry DOI information from the multiple timestamps that are generated per gamma event. The difference between two timestamps that are triggered by scintillation photons traveling in opposite directions signifies the event's depth-dependent optical photon TOF (oTOF). However, light leak at the crystal-readout interface substantially degrades the resolution of this oTOF-based depth encoding.

The feasibility of oTOF-based depth encoding was demonstrated by mitigating light leak in single-ended-readout Prism-PET detector modules using tapered crystals. Minimizing light leak also improved both energy-based DOI and coincidence timing resolutions.

The tapered Prism-PET module may include a 16×16 array of 1.5×1.5×20 mm³ lutetium yttrium oxyorthosillicate (LYSO) crystals, which are tapered down to 1.2×1.2 mm² at the crystal-readout interface. The LYSO array couples 4-to-1 to an 8×8 array of 3×3 mm² silicon photomultiplier (SiPM) pixels on the tapered end and to a segmented prismatoid light guide array on the opposite end. Performance of tapered and non-tapered Prism-PET detectors was experimentally characterized and evaluated by measuring flood histogram, energy resolution, energy- and oTOF-based DOI resolutions, and coincidence timing resolution. Sensitivities of scanners using different Prism-PET detector designs were simulated using Geant4 application for tomographic emission (GATE).

For the tapered (non-tapered) Prism-PET module, the measured full width at half maximum (FWHM) energy, timing, energy-based DOI, and oTOF-based DOI resolutions were 8.88 (11.18) %, 243 (286) ps, 2.35 (3.18) mm, and 5.42 (13.87) mm, respectively. The scanner sensitivities using non-tapered and tapered crystals, and 10 rings of detector modules, were simulated Date/Am to be 30.9 and 29.5 kcps/MBq, respectively. The tapered Prism-PET module with minimized light leak enabled the first experimental report of oTOF-based depth encoding at the detector module level. It also enabled the utilization of thinner (i.e., 0.1 mm) inter-crystal spacing with barium sulfate as the reflector while also improving energy-based DOI and timing resolutions.

### 1 Introduction

Positron emission tomography (PET) has tremendous potential for early detection of brain disorders before the symptomatic onset and accurate differential diagnosis of psychiatric disorders or neurodegenerative diseases such as Alzheimer's disease (AD) due to its distinctive ability to directly image molecular markers of neuropathology. PET is also playing a burgeoning role in neuro-oncology with emerging radiotracers for noninvasive grading of primary brain tumors, delineation of tumor extent, differentiation of tumor recurrence from treatment-related changes, surgical or radiotherapeutic planning, assessment of response, and post-treatment monitoring. However, low spatial resolution in PET limits the quantitative accuracy of small subcortical nuclei such as locus coeruleus (LC) which has evident neuropathological changes in the early stages of AD, and partial volume effect degrades the measurements of tumor uptake. Thus, the improvement in spatial resolution is necessary for reliable quantitative PET neuroimaging.

One of the fundamental limits for spatial resolution is annihilation photon acollinearity, which can be minimized by a small-diameter and conformal brain PET scanner that arranges detectors close to the subject. In addition, the compact and conformal geometry design not only reduces costs by using fewer detectors but also increases sensitivity by providing a larger solid angle coverage when compared to whole-body cylindrical PET scanners. Nevertheless, such a geometry causes substantial image blur due to parallax error not only at the peripheral but also at the center of the field-of-view (FOV). Thus, DOI is an indispensable capability for the PET detector module to correct PE and attain uniform spatial resolution across the FOV. Various DOI detectors have been developed in recent years, including dual-ended readout detectors that can resolve small crystals but increase the cost by using two photodetectors per detector module, monolithic scintillator detectors that have higher intrinsic spatial resolution but suffer from time-consuming calibration procedures and complex positioning algorithms, and single-ended readout detector with light-sharing and segmented crystal array that achieves high resolution at a low cost. A practical single-ended DOI-encoding detector module that utilizes a cuboid crystal array and a segmented prismatoid light-guide array, hence the name Prism-PET, for enhanced and localized light-sharing was recently proposed. The first compact and conformal brain prototype scanner based on the Prism-PET detectors has obtained the highest-resolution PET phantom images and enabled accurate visualization as well as uptake quantification of small brain nuclei.

The effective sensitivity gain due to TOF is estimated as:

$${TOF}_{gain} = \sqrt{{α}^{2} \frac{2 D}{c Δ t}} = \sqrt{{α}^{2} \frac{D}{Δ x}} , \qquad ( 1 )$$

where α is a factor related to image reconstruction, D is object size, c is the speed of light, Δt is TOF resolution, and Δx, the uncertainty in spatial localization along the LOR, is determined by the TOF resolution. This gain is reduced for brain-dedicated PET systems, compared to whole-body scanners, due to the smaller object size, and thus, better TOF resolution is needed to compensate for the smaller D. Reaching a high TOF gain requires mitigating the DOI-induced bias on timing resolution (in the order of 150-200 ps) since coincidence events at different DOIs can lead to biased estimation of photon arrival times, especially for long crystals (15-25 mm). Multiple approaches have been explored over the years to incorporate the DOI-induced error into the analytical modeling of timing resolution in PET detectors. The contribution to mitigate this degrading effect is integrating the DOI-dependent timestamps correction into the calculation of timing resolution which resulted in an enhanced timing resolution for the Prism-PET prototype scanner.

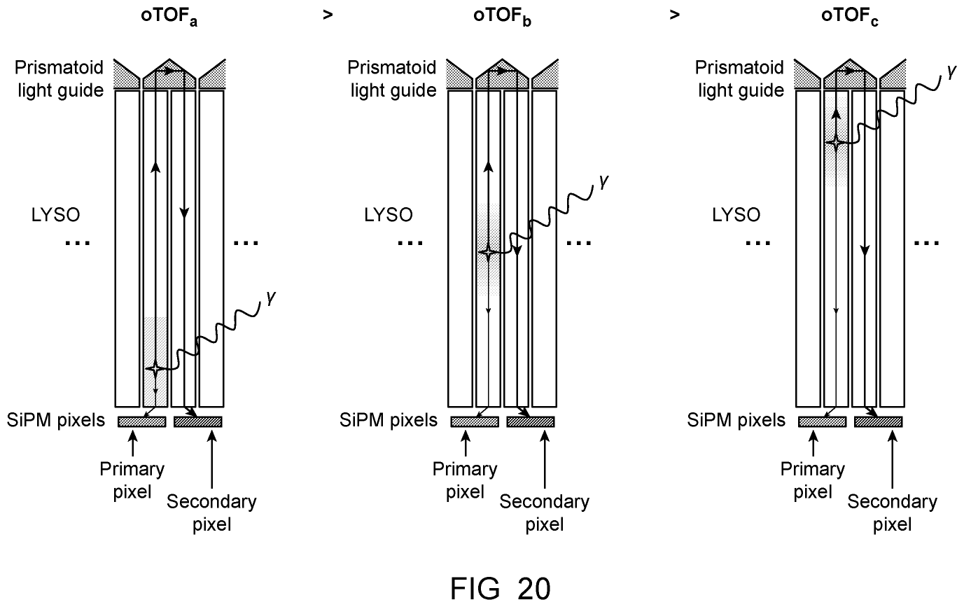

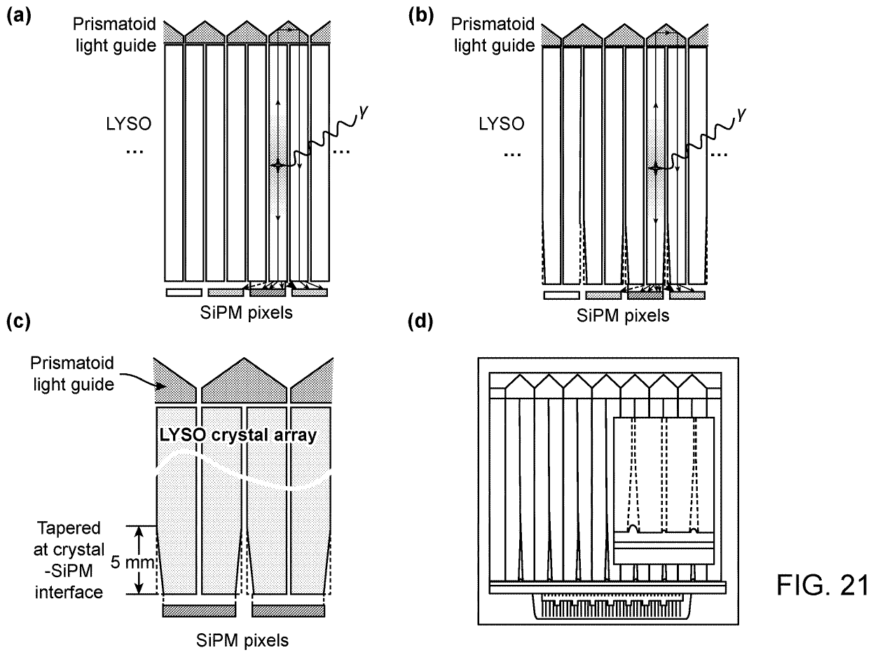

When scintillation photons are generated within a crystal column in a Prism-PET detector module, some photons travel downward to trigger the primary SiPM (i.e., primary pixel) and some travel upward to the light guide which are then steered to the nearest-neighboring crystals and travel downward to trigger the nearest-neighboring SiPMs (i.e., secondary pixels, see FIG. 20). Ideally, single-ended light-sharing PET modules with TOF information (such as Prism-PET) should be able to estimate DOI using the optical photon TOF (oTOF) which is the difference between the primary and secondary timestamps. DOI information is intrinsically encoded in multiple light-sharing timestamps with the smallest oTOF due to almost simultaneous triggering of primary and secondary SiPMs for photon interaction close to the light guide, and the largest oTOF due to photon interaction close to the crystal-SiPM interface (FIG. 20). However, oTOF-based depth encoding has not yet been reported experimentally due to light leak which causes a SiPM to be triggered by photons from neighboring crystal and not the crystal that it is coupled to. Light leak is caused by 1) the finite gap between crystal and SiPM, which is the summation of the thickness of the coupling glue and protective resin (grey arrow in FIG. 21(a)), and 2) when the crystal column extends beyond the pixel's active area (green arrow in FIG. 21(a)).

In this paper, a next-generation Prism-PET detector module was developed using unilaterally tapered crystals to minimize the scintillation light leak at the crystal-readout interface (FIG. 21(b)), improve both timing and DOI resolutions and enable the potential utilization of the novel oTOF-based DOI estimation methods. Here the tapered and non-tapered (cuboid) Prism-PET module performance was experimentally characterized in terms of flood histogram, energy resolution, DOI resolutions (weighted and combined methods), and timing resolution. Furthermore, sensitivities of 10-ring Prism-PET scanners with tapered and cuboid modules were evaluated in Geant4 application for tomographic emission (GATE) simulation.

### 2 Materials and Methods

### 2.1 Tapered Prism-PET Detector Module

The tapered TOF-DOI Prism-PET detector module developed in this study comprised of a 16×16 array of 1.5×1.5×20 mm³ lutetium yttrium oxyorthosillicate (LYSO) crystals (Shanghai EBO Optoelectronic Technology CO., China) which were tapered down to 1.2×1.2 mm² at the crystal-readout interface, as shown in FIG. 21. The tapered LYSO array coupled 4-to-1 to an 8×8 array of 3×3 mm² silicon photomultiplier (SiPM) pixels (Hamamatsu Photonics K.K., Japan) on the tapered side and to a prismatoid light guide array on the opposite side. The segmented light guide was made of an array of right triangular prisms with three unique designs at the center, edge, and corner, which efficiently redirected scintillation photons to only the nearest neighboring SiPMs and thus enabled enhanced and localized light sharing. Barium sulfate (BaSO₄) was used as the reflector material between the crystals and prisms to ensure optical isolation.

### 2.2 Data Acquisition and Calibration

Data acquisition was implemented by using a TOFPET2 application-specific integrated circuits (ASICs) evaluation kit which includes a TOF front-end board (FEB/D) and two front-end modules (FEM), with each FEM containing four TOFPET2 ASIC that read 256 channels. SiPMs in the tapered module were read out by a TOFPET2 ASIC with 64 channels, and the signal from each channel was fed into an analog-to-digital converter (ADC) as well as a time-to-digital converter (TDC) to acquire energy and timing information, respectively. To evaluate whether the incoming signal is valid, three discriminator thresholds were utilized in the evaluation kit including one energy threshold (vth_e) and two timing thresholds (vth_t1 and vth_t2) . . . . All ASICs in the evaluation kit were calibrated using the calibration routine in the PETsys software before any data acquisition. All acquired list-mode data was processed with an energy window of 460-560 keV, a coincidence time window of 15 ns, and inter-crystal scatter (ICS) rejection. Given that ASIC circuitry's calibration is affected by large temperature variances, all chips were maintained within 20±0.2° C. by conducting experiments in a temperature-controlled chamber.

### 2.3 Flood Histogram

The measurement was performed by uniformly irradiating the tapered Prism-PET module with a 1-mm diameter ²²Na point source positioned 10 cm away from the module. The same measurements were implemented using the two previously designed 4-to-1 coupled Prism-PET modules for comparison: one with 1.4×1.4×20 mm³ LYSO crystals (1.4-mm cuboid Prism-PET module) and the other with 1.5×1.5×20 mm³ LYSO crystals (1.5-mm cuboid Prism-PET module). The two-dimensional (2D) coordinates of flood histogram were calculated from the acquired list-mode data using a truncated center of gravity (tCoG) method. The flood histogram was segmented using k-means clustering and corrected by moving the center of each segmented cluster to its corresponding equally sampled crystal region.

### 2.4 Energy Resolution

Crystal look-up table (LUT) was generated from the corrected flood histogram and used to obtain the energy spectrum for each crystal. The energy spectrum was fitted into a Gaussian function with its full width at half maximum (FWHM) value representing the crystal energy resolution. The detector module energy resolution was the average energy resolution of all crystals.

### 2.5 DOI Resolution

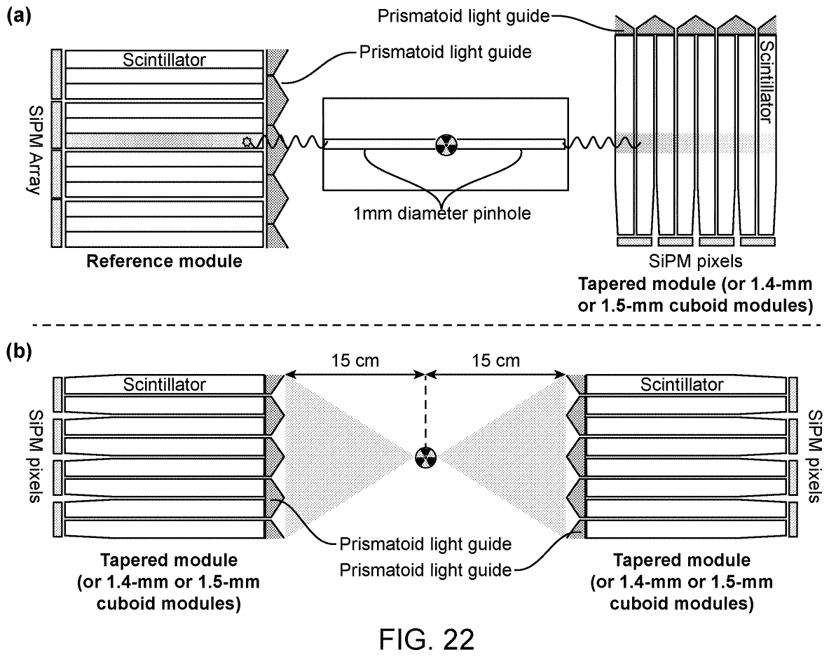

The DOI measurements were performed in coincidence mode using a Prism-PET detector module including a 24×24 array of 0.9×0.9×20 mm³ LYSO crystals coupled 9-to-1 to an 8×8 array of SiPMs as a reference module. The tapered Prism-PET module was selectively irradiated at 19 depths ranging from 1 mm to 19 mm with a step size of 1 mm. A lead collimation was used with the same ²²Na point source placed at the center of the lead cylinder with a 1-mm pinhole. The schematic of the experimental setup for the DOI measurements is shown in FIG. 22(a). Only coincidence events occurring between the tapered module and the crystal with the highest counting statistics in the reference module were accepted in order to reject Compton scatter. While previous works utilized a single scintillator with SiPM as the reference crystal, A reference crystal was used from the 9-to-1 Prism-PET module to simplify the alignment process to the collimator pinhole (see Ref. [25] for the details of experiment setup).

### 2.5.1 Weighted Method

Two weighted methods were used to estimate DOI variables for the 1.5-mm tapered Prism-PET module. (1) energy-weighted DOI variable is derived as:

$${w}_{E} = \frac{{E}_{max}}{E} , \qquad ( 2 )$$

where Eₘₐₓ is the maximum intensity signal from the primary SiPM, and E is the total detected signals from the primary and light-sharing SiPMs. (2) oTOF-weighted DOI variables is estimated as:

$${w}_{oTOF} = \frac{{\sum }_{i = 1}^{n} {oTOF}_{i}}{n} = \frac{{\sum }_{i = 1}^{n} ( {t}_{i} - {t}_{p} )}{n} , \qquad ( 3 )$$

where tₚ is the timestamp generated by the primary SiPM pixel and tᵢ is the nearest-neighbor light-sharing timestamp (LSTS) generated by the secondary SiPM pixel, and n is 3, 2, and 1 for center, corner, and edge crystals, respectively. The w histograms at five depths (2, 6, 10, 14, and 18 mm) were converted to DOI histograms using linear regression to calculate the slope between w and the ground-truth DOI. DOI distributions were then fitted into Gaussian functions and their FWHM values represented DOI resolutions. Linear regression analysis was implemented to calculate the correlation between w_{E} and w_{TOF} parameter. The same DOI measurements were performed using 1.4-mm and 1.5-mm cuboid modules for comparison.

### 2.5.2 Combined Method

The energy-weighted DOI and oTOF-weighted DOI methods were combined to estimate the DOI of all events at 5 depths. The resultant DOI (DOI_{c}) is given as:

$${DOI}_{c} = {c}_{1} \cdot {DOI}_{E} + {c}_{2} \cdot {DOI}_{oTOF} , \qquad ( 4 )$$

where the constants c₁ and c₂ represent the weighting coefficients that their sum equals unity, DOIE is DOI value calculated using the energy information, and DOI_{oTOF} is DOI value estimated using oTOF information. Six different combinations of c₁ and c₂ are employed in the equation, with ratios r=c₂/c₁ selected as 0/1, 0.1/0.9, 0.2/0.8, 0.3/0.7, 0.4/0.6, and 0.5/0.5. The DOI_{c} histograms of all depths were plotted and fitted into Gaussian functions with their average FWHM values representing DOI resolution for each r.

### 2.6 Timing Resolution

To evaluate timing resolution, a 0.25-mm ²²Na point source with radioactivity of 1.85 MBq was positioned at the center of pairs of 1.4-mm cuboid, 1.5-mm cuboid, and 1.5-mm tapered Prism-PET modules. FIG. 22(b) shows the schematic of the experimental setup for the timing measurement. The acquired point source data was processed using the time offset correction of three-dimensional (3D) channel ID represented by the 2D crystal ID (radial number, axial number) as well as the DOI bin number. The timestamps for each gamma interaction were also corrected with primary and nearest-neighbor LSTSs, thanks to the enhanced and localized light sharing in Prism-PET which generates a characteristic pattern of SiPM signals. Besides, a LOR-based finetuning step was employed to optimize timing resolution further. The timing resolution was measured as the FWHM of the Gaussian-fitted timing spectrum.

### 2.7 Sensitivity of Simulated Scanners

The sensitivities of a 10-ring conformal Prism-PET brain scanner (a long diameter of 38.5 cm, a short diameter of 29.1 cm, and an axial length of 26.4 cm) using the 1.4-mm and 1.5-mm cuboid detector modules were first evaluated using GATE simulation. A 70-cm line source with 1-mm diameter was filled with 1 MBq ¹⁸F and inserted inside a set of five concentric 70-cm aluminum sleeves with known diameters to estimate attenuation-free sensitivity. This line source phantom was placed at the center of the FOV and five simulations (starting with all five aluminum sleeves and removing one sleeve each time) were performed for 100 s each. The energy window, energy resolution, and coincidence time window were set to 450-650 keV, 10%, and 2.5 ns, respectively. To evaluate the sensitivity of the 10-ring Prism-PET brain scanner with tapered modules, acquired list-mode data from simulations of the scanner with 1.5-mm cuboid modules were also processed to filter the events that occurred outside the tapered crystals. Axial sensitivity profiles and system sensitivities, based on the National Electrical Manufacturers Association (NEMA) NU2-2018 guidelines, were obtained using a single-slice rebinning (SSRB) method for original simulation data (scanners with cuboid modules) and truncated simulation data (scanner with tapered modules).

### 3 Results

### 3.1 Flood Histogram and Energy Resolution

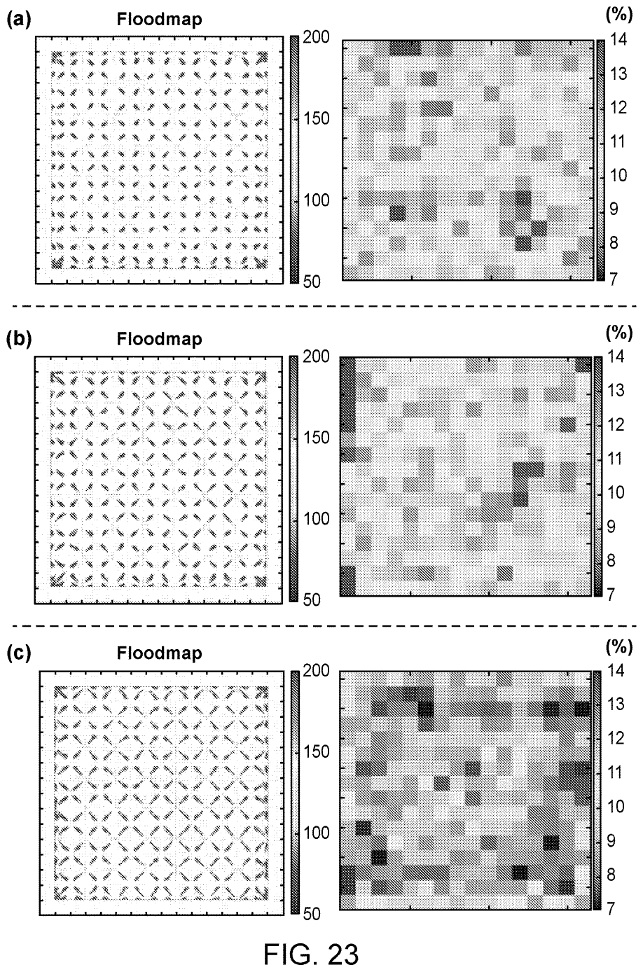

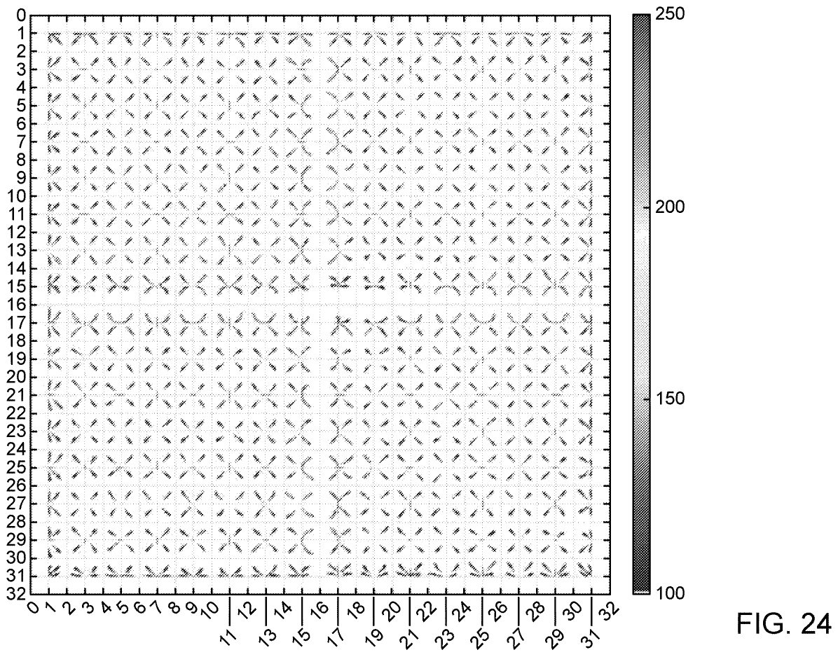

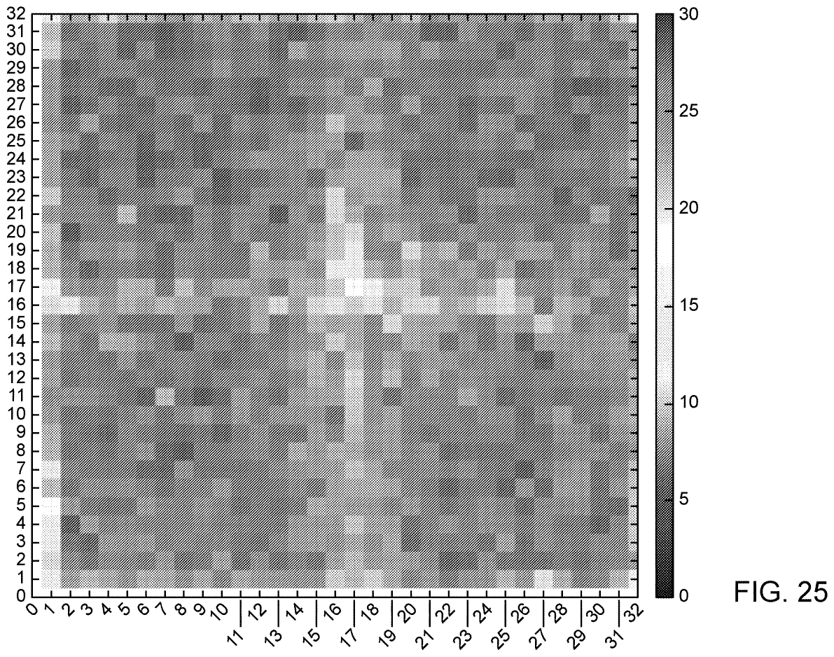

The flood histograms and energy resolutions of each crystal for the 1.4-mm cuboid, 1.5-mm cuboid, and 1.5-mm tapered Prism-PET modules are shown in FIG. 23. Excellent crystal identification was achieved for the entire LYSO array in the tapered module, demonstrating that the decoding error was negligible. The average energy resolutions across all crystals in the 1.4-mm cuboid, 1.5-mm cuboid, and 1.5-mm tapered modules were 10.94±1.21%, 11.18±1.37%, and 8.88±1.03%, respectively (uncorrected for saturation). The tapered Prism-PET module achieved the best energy resolution compared to the cuboid Prism-PET modules. The flood histograms and energy resolutions of each crystal for the 3.0 mm tapered Hexagonal-PET modules are shown in FIGS. 23-25. All the center, edge, and corner crystals can be clearly resolved, indicating the decoding error of the detector is negligible. The average energy resolution across all crystals in the 3.0 mm tapered module is 7.76±1.18%. Therefore, the mm tapered Hexagonal-PET modules achieved the best energy resolution compared to the cuboid Prism-PET modules and the tapered Prism-PET modules.

### 3.2 DOI Resolution

### 3.2.1 Weighted Method

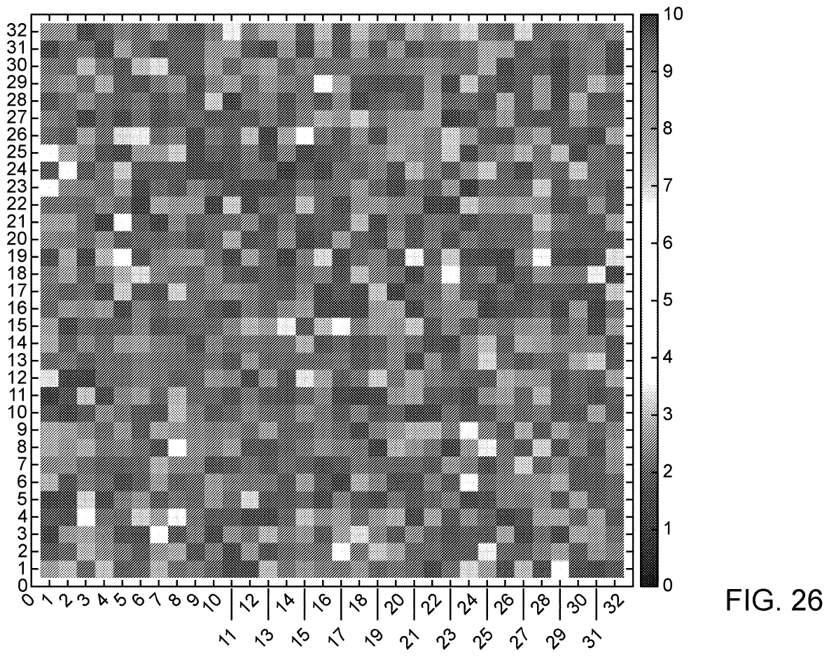

FIG. 26 shows resolutions for 1024 crystals of the Hexagonal-PET module. The DOI resolution across all crystals is 2.12±1.25 mm full-width-at-half-maximum (FWHM). Two methods can be used to estimate DOI variables for each crystal in the detector module. (1) energy-weighted DOI variable is derived as:

$${w}_{E} = \frac{{E}_{max}}{E}$$

where Eₘₐₓ is the maximum intensity signal from the primary SiPM, and E is the total detected signals from the primary and light-sharing SiPMs.
(2) oTOF-weighted DOI variables is estimated as:

$${w}_{oTOF} = \frac{{\sum }_{i = 1}^{n} {oTOF}_{i}}{n} = \frac{{\sum }_{i = 1}^{n} ( {t}_{i} - {t}_{p} )}{n}$$

where tₚ is the timestamp generated by the primary SiPM pixel and tᵢ is the nearest-neighbor light-sharing timestamp (LSTS) generated by the secondary SiPM pixel, and n is 3, 2, and 1 for center, corner, and edge crystals, respectively. Two different ways can be used to calculate the DOI resolution of the detector: (1) selecting the best DOI resolution between energy-based and oTOF-based method for each crystal, and subsequently averaging the DOI resolutions across all 1024 crystals; (2) performing a weighted averaging of the energy-based and oTOF-based DOI variables for each crystal to determine a new DOI resolution for each crystal, followed by averaging the DOI resolutions across all 1024 crystals.

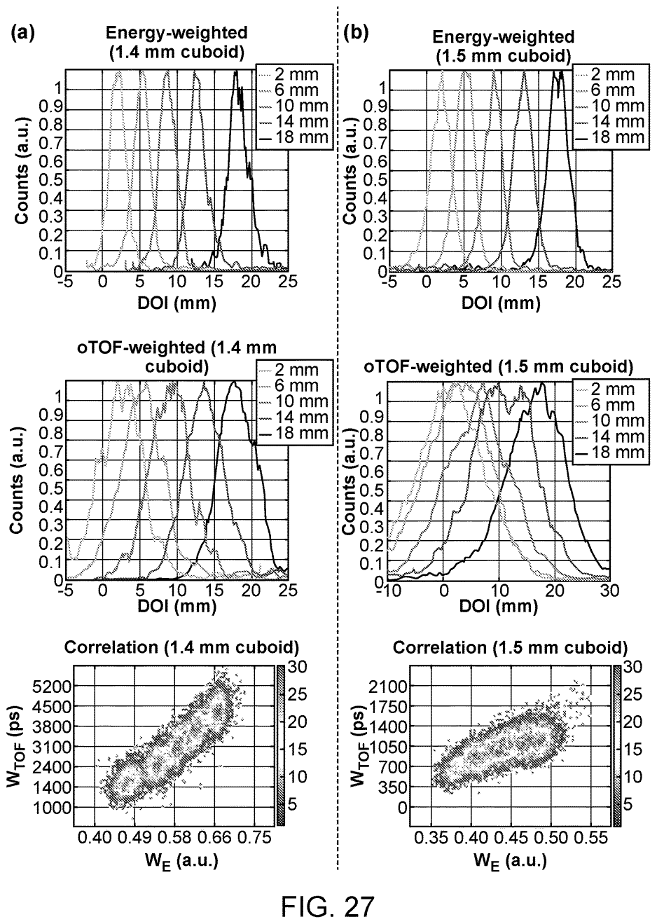

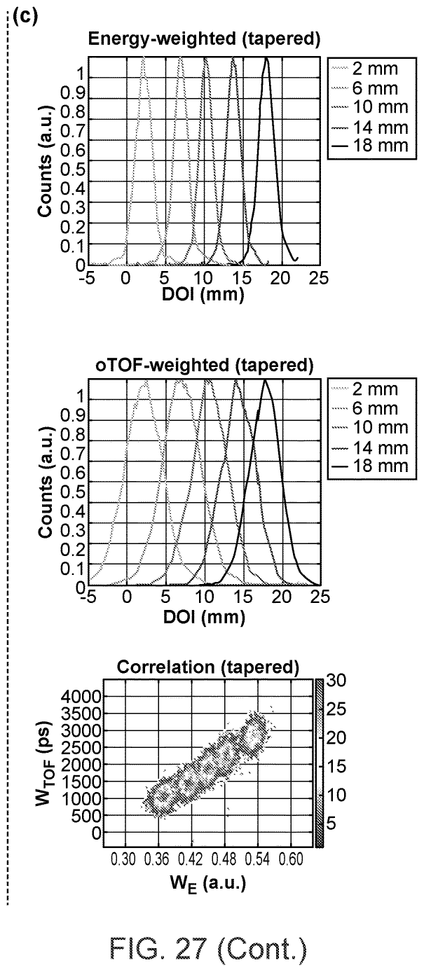

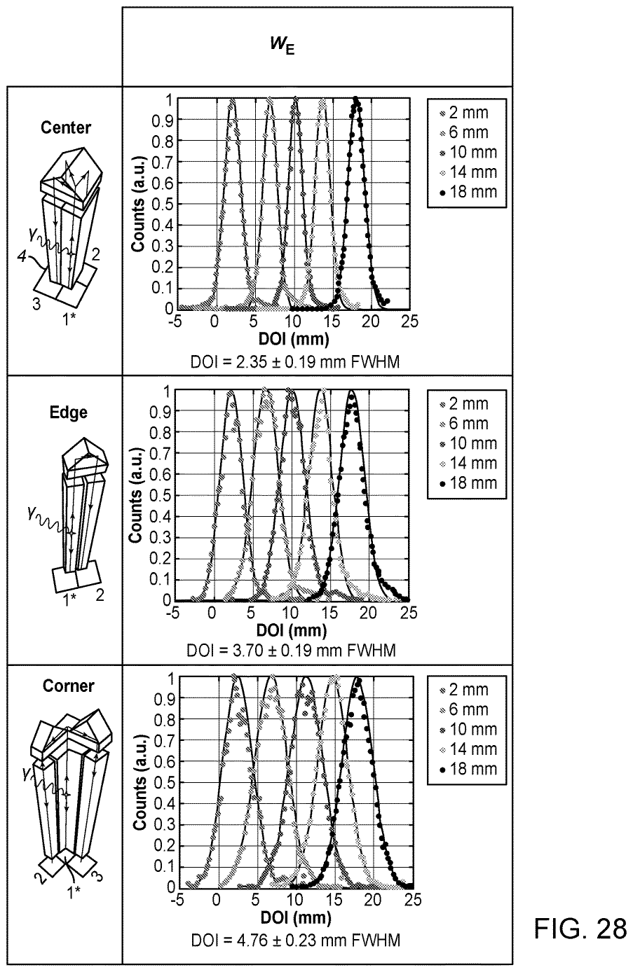

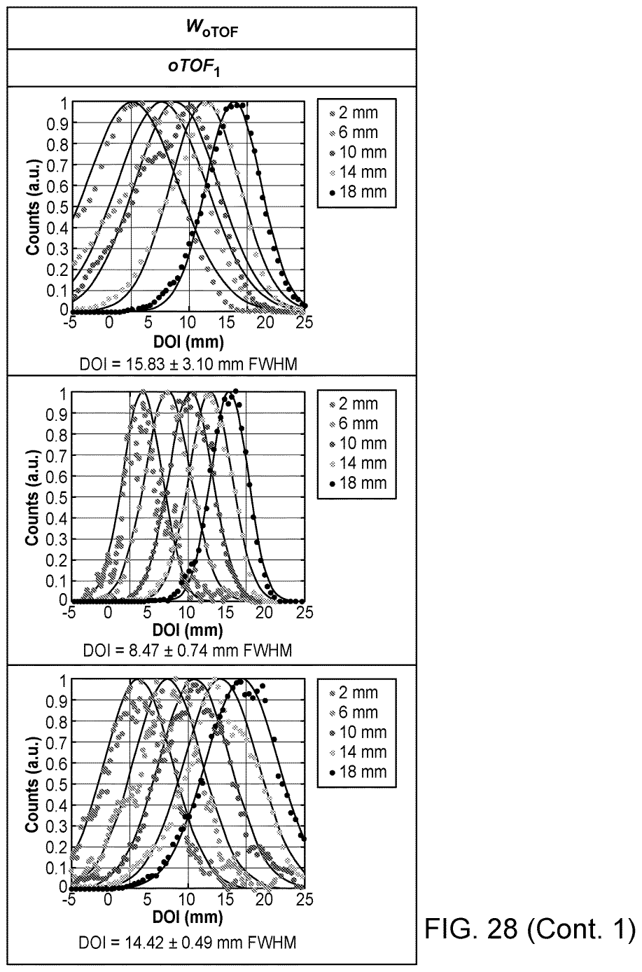

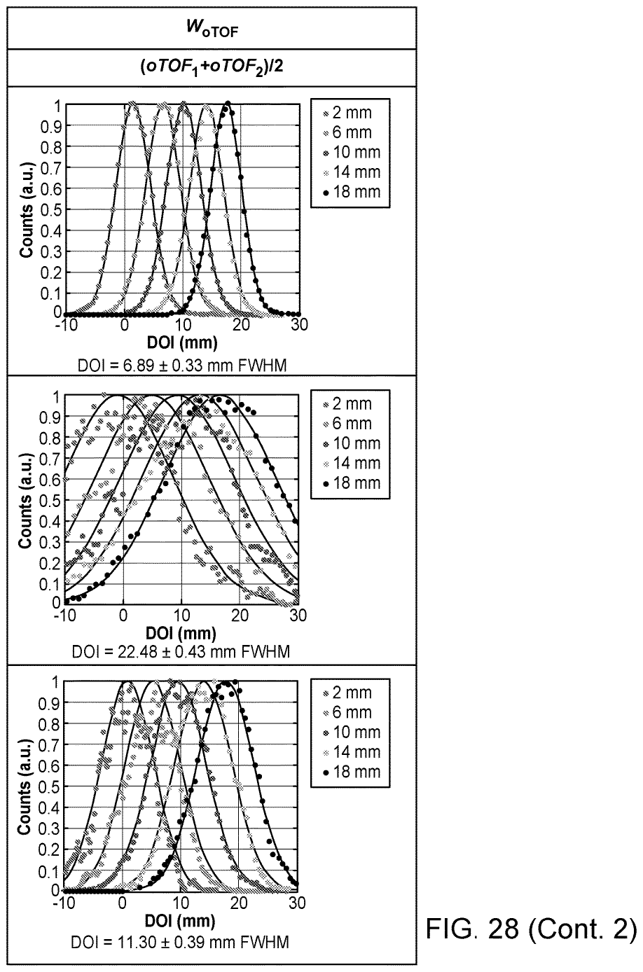

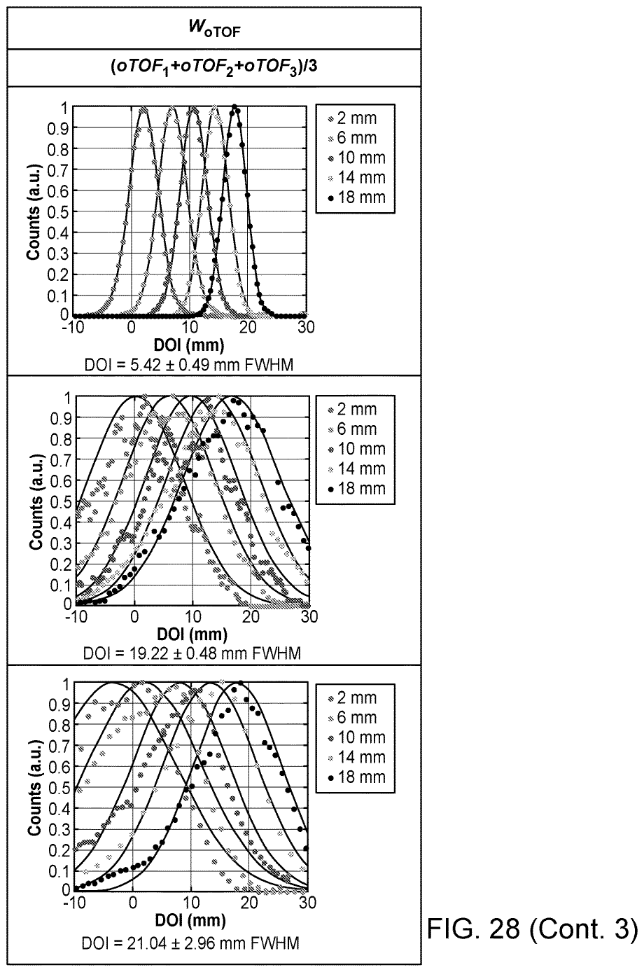

FIG. 27 shows energy-weighted (w_{E}) DOI distributions, oTOF-weighted (w_{TOF}) DOI distributions, and correlation between w_{E} and w_{TOF} for center crystals in cuboid and tapered Prism-PET modules at depths of 2, 6, 10, 14, and 18 mm. The average w_{E} DOI resolutions across five depths were 2.59±0.36 mm, 3.18±0.23 mm, and 2.35±0.19 mm FWHM for the center crystals in 1.4-mm cuboid, 1.5-mm cuboid, and 1.5-mm tapered Prism-PET modules, respectively. The corresponding average w_{TOF} DOI resolutions were 6.45±0.51 mm, 13.87±0.87 mm, and 5.42±0.49 mm FWHM, respectively. The correlations were 0.80 for the 1.4-mm cuboid module, 0.43 for the 1.5-mm cuboid module, and 0.83 for the tapered module. The tapered module achieved the best DOI resolutions and the highest correlation, while the poorest DOI resolutions were obtained for the 1.5-mm cuboid module. FIG. 28 shows DOI distributions of w_{E} and three w_{TOF} for center, edge, and corner crystals of the tapered module at five depths. The DOI resolutions using the classical w_{E} method were 2.35±0.19 mm, 3.70±0.19 mm, and 4.76=0.23 mm FWHM for center, edge, and corner crystals, respectively. The best DOI resolution of 5.42±0.49 mm FWHM was obtained using (oTOF₁+oTOF₂+oTOF₃)/3 for the center scenario incorporating three nearest-neighbor SiPM pixels. Optimal DOI resolutions of 8.47±0.74 mm and 11.30±0.39 mm were attained using oTOF₁ and (oTOF₁+oTOF₂)/2 for the edge scenario with only one nearest-neighbor SiPMs and the corner scenario with two nearest-neighbor SiPMs, respectively. The tapered module's center, edge, and corner crystals attained their best w_{oTOF} DOI resolutions by employing three, one, and two oTOF values, respectively, validating the effectiveness of three distinct light-sharing designs. By weighting the DOI resolutions based on the percentage of crystals in each region within the crystal array (76.56% center, 21.88% edge, and 1.56% corner), averaged DOIw_{E} and DOIw_{TOF} resolutions of 2.68 mm and 6.18 mm FWHM were achieved for tapered Prism-PET module.

### 3.2.2 Combined Method

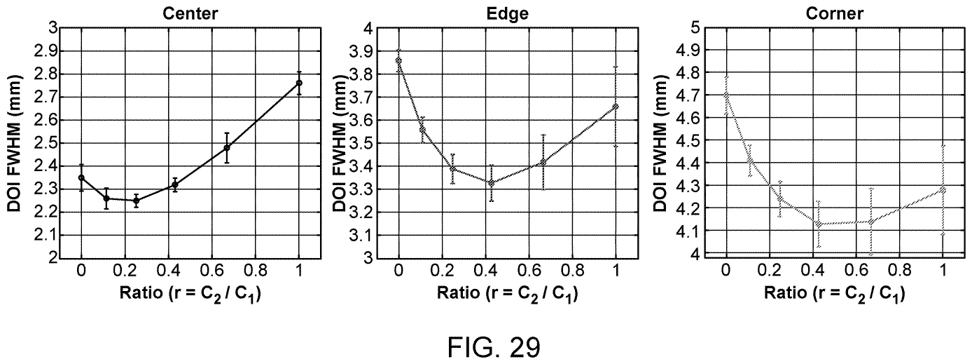

The combined DOI resolutions of six ratios (r=0/1, 0.1/0.9, 0.2/0.8, 0.3/0.7, 0/4/0.6, and 0.5/0.5) for center, edge, and corner crystals are shown in FIG. 29. An optimal DOI resolution of 2.25±0.14 mm was achieved when employing r=0.2/0.8 for the center crystal. At r=0.3/0.7, the edge and corner crystals demonstrated their best DOI resolutions of 3.33=0.39 mm and 4.13±0.50 mm, respectively. After weighing the best DOI resolutions of center, edge, and corner crystals, the average DOI resolution of the tapered module was 2.52 mm.

### 3.3 Timing Resolution

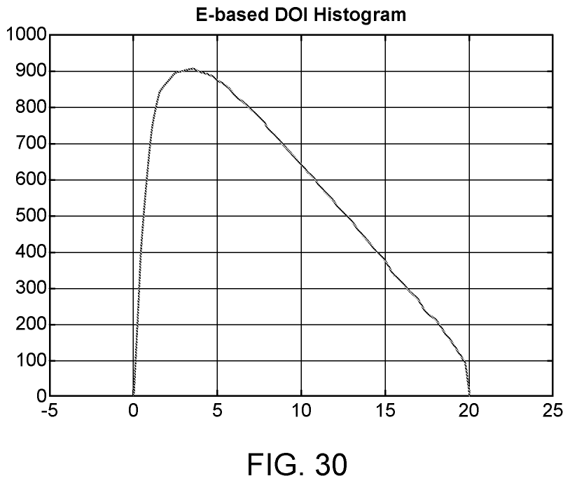

Timing spectra with the ²²Na point source placed at the center of pairs of 1.4-mm cuboid modules, 1.5-mm cuboid modules, and 1.5-mm tapered modules are shown in FIG. 30. The timing resolutions were 254 ps, 286 ps, and 243 ps FWHM utilizing the time offset and LSTS corrections of 3D channels together with LOR-based fine-tuning for the 1.4-mm cuboid, 1.5-mm cuboid, and tapered modules, respectively.

### 3.4 Sensitivity of Simulated Scanners

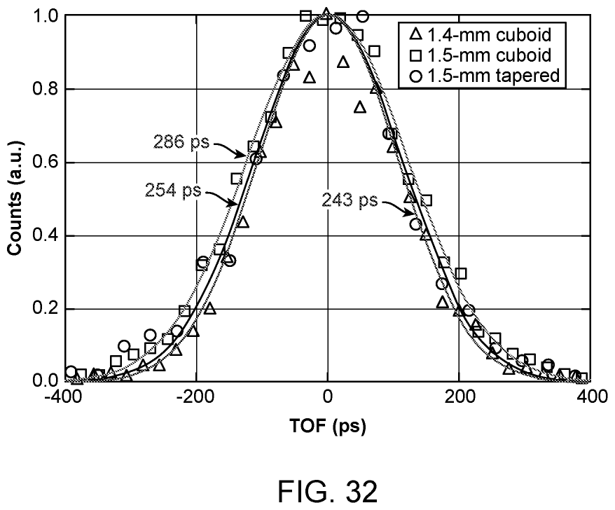

The axial sensitivity profiles of the 10-ring Prism-PET scanner with 1.4-mm cuboid, 1.5-mm cuboid, and tapered modules are shown in FIG. 32. The corresponding system sensitivities extrapolated from five data sets (beginning with five aluminum sleeves and finishing with a single sleeve) were 25.0, 30.9, and 29.5 kcps/MBq, respectively.

### 4 Discussion

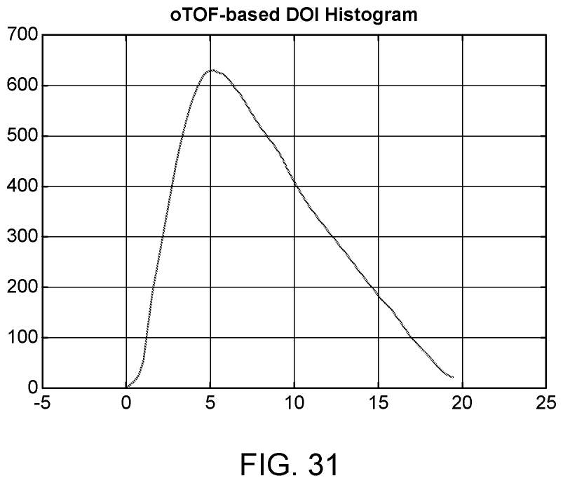

Originally, a crystal cross-sectional area (CSA) and inter-crystal reflector thickness of 1.4×1.4 mm² and 0.2 mm, respectively, were used. However, to increase detector efficiency (DE), the design was changed to a crystal CSA and inter-crystal reflector thickness of 1.5×1.5 mm² and 0.1 mm, respectively. In a 4-to-1 coupled Prism-PET module (i.e., four crystals overlaying one 3×3 mm² SiPM pixel) with cuboid crystals (called cuboid Prism-PET), this new design caused each crystal to exceed the pixel's active area on 2 perpendicular sides by 0.05 mm which exacerbated the scintillation light leak and consequently degraded the overall DOI resolution of the detector module by ~20% compared to that of a cuboid Prism-PET module with the original design (see FIG. 27). Thus, the tapered crystal design was used (FIG. 21) in a Prism-PET module (called tapered Prism-PET) to minimize scintillation light leak at the crystal-SiPM interface and enhance the DOI resolution. Minimizing light leak using tapered crystals and maximizing localized light sharing using segmented prismatoid light guides enabled depth-encoding using scintillation photon TOF (or oTOFs), which are the difference between the primary timestamp (triggered by scintillation photons traveling downward to the primary SiPM) and nearest-neighbor light sharing timestamps (triggered by scintillation photons traveling upward to the light-guide and steered downward to the secondary SiPMs). Given that both energy values and timestamps are provided in list-mode by the PETsys readout, energy-based DOI (DOI_{E}) and oTOF-based (DOI_{oTOF}) were weighted averaged to improve the DOI localization accuracy with the optimal weighting factors c₂/c₁ being 0.2/0.8 (i.e., r=0.25) for center crystals and 0.3/0.7 (i.e., r=0.43) for edge and corner crystals (FIG. 29). FIG. 30 and FIG. 31 depicts an example histogram of results of the energy-based DOI (DOI_{E}) and the oTOF-based (DOI_{oTOF}), respectively.

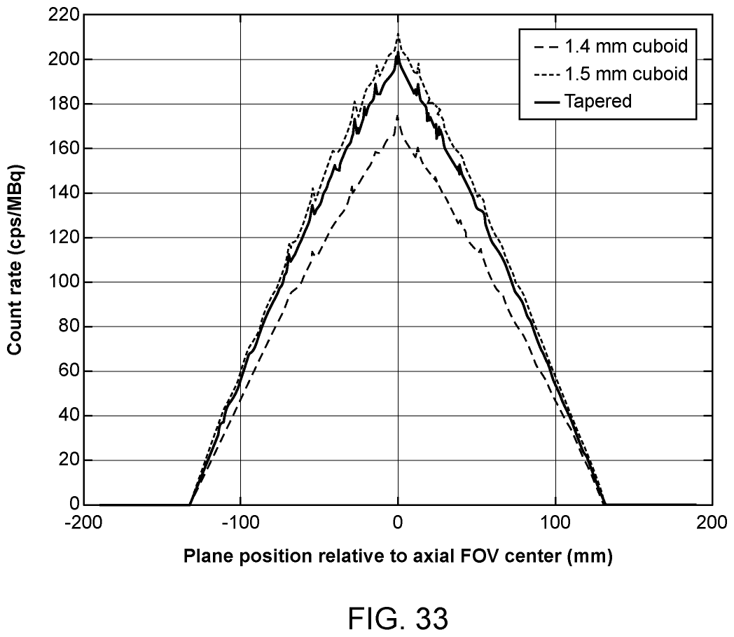

In addition, the degradation in DE due to tapered crystals is very small because gamma photon interaction follows the Beer-Lambert law and the small volume reduction due to tapering is at the tail of the exponential distribution. The system sensitivity of the 10-ring Prism-PET scanner. with 1.5-mm tapered crystals is 4.5% lower and 15.3% higher than that of the scanners with 1.5-mm and 1.4-mm cuboid crystals, respectively. Apart from reporting the best DOI resolution among other depth encoding detectors with single-ended readout, the 1.5-mm tapered Prism-PET module also achieved enhanced energy resolution (FIG. 32) and timing resolution (FIG. 33) due to substantial reduction in light leak. More specifically, improvement in timing resolution is because more scintillation photons that travel downward towards the primary SiPM are in fact detected by the primary SiPM and not lost due to inter-pixel gap or crosstalk. Note that timing resolution can be further improved by optimizing the rising edge discriminator threshold and overvoltage of PETsys TOFPET2 ASIC.

### 5 Conclusion

The tapered Prism-PET modules with minimized light leak 1) enabled the utilization of thinner (i.e. 0.1 mm) inter-crystal spacings (filled with BaSO₄ reflectors) and 1.5×1.5 mm² crystal CSA for enhanced sensitivity, 2) improved energy-based DOI localization, and 3) to the best knowledge, enabled the first experimental report of oTOF-based depth encoding at the detector module level. For future work, 1.5 mm tapered Prism-PET detector modules will be utilized with the recently developed interleaved multiplexing (iMUX) readout to extend the axial FOV of the conformal brain-dedicated PET scanner for high-resolution PET neuroimaging in human subjects.

Having now described some illustrative implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements, and features discussed in connection with one implementation are not intended to be excluded from a similar role in other implementations or implementations.

The hardware and data processing components used to implement the various processes, operations, illustrative logics, logical blocks, modules, and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general-purpose processor may be a microprocessor, or any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device, etc.) may include one or more devices (e.g., RAM, ROM, Flash memory, hard disk storage, etc.) for storing data and/or computer code for completing or facilitating the various processes, layers, and modules described in the present disclosure. The memory may include volatile memory or non-volatile memory, and may include database components, object code components, script components, or any other type of information structure for supporting the various activities and information structures described in the present disclosure. According to an embodiment, the memory is communicably connected to the processor via a processing circuit and includes computer code for executing (e.g., by the processing circuit and/or the processor) the one or more processes described herein.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising of machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general-purpose computer, special purpose computer, or special purpose processing machines to perform a certain function or group of functions.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation or embodiment, and references to “an implementation,” “some implementations,” “one implementation” or the like are not necessarily mutually exclusive and are intended to indicate that a particular feature, structure, or characteristic described in connection with the implementation can be included in at least one implementation or embodiment. Such terms as used herein are not necessarily all referring to the same implementation. Any implementation can be combined with any other implementation, inclusively or exclusively, in any manner consistent with the aspects and implementations disclosed herein.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements. Technical and scientific terms used herein have the meanings commonly understood by one of ordinary skill in the art, unless otherwise defined. Any suitable materials and/or methodologies known to those of ordinary skill in the art can be utilized in carrying out the methods described herein.

Systems and methods described herein may be embodied in other specific forms without departing from the characteristics thereof. As used herein, “approximately,” “about” “substantially” or other terms of degree will be understood by persons of ordinary skill in the art and will vary to some extent on the context in which it is used. If there are uses of the term which are not clear to persons of ordinary skill in the art given the context in which it is used, references to “approximately,” “about” “substantially” or other terms of degree shall include variations of +/−10% from the given measurement, unit, or range unless explicitly indicated otherwise.

Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

The term “coupled” and variations thereof includes the joining of two members directly or indirectly to one another. Such joining may be stationary (e.g., permanent or fixed) or moveable (e.g., removable or releasable). Such joining may be achieved with the two members coupled directly with or to each other, with the two members coupled with each other using a separate intervening member and any additional intermediate members coupled with one another, or with the two members coupled with each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References to “or” can be construed as inclusive so that any terms described using “or” can indicate any of a single, more than one, and all of the described terms. A reference to “at least one of ‘A’ and ‘B’” can include only ‘A’, only ‘B’, as well as both ‘A’ and ‘B’. Such references used in conjunction with “comprising” or other open terminology can include additional items.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

References herein to the positions of elements (e.g., “top,” “bottom,” “above,” “below”) are merely used to describe the orientation of various elements in the FIGURES. The orientation of various elements may differ according to other exemplary embodiments, and that such variations are intended to be encompassed by the present disclosure.

As used herein, a subject can be a mammal, such as a non-primate (e.g., cows, pigs, horses, cats, dogs, rats, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets. In certain exemplary embodiments, a subject is a human.

As used herein, the terms “subject” and “user” are used interchangeably. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

As used herein, the singular forms “a”, “an,” and “the” include plural referents unless the context clearly indicates otherwise. For example, the term “a cell” includes a plurality of cells, including mixtures thereof.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 5 should be considered to have specifically disclosed subranges such as from 1 to 3, from 1 to 4, from 1 to 5, from 2 to 4, from 2 to 5, from 3 to 5, etc., as well as individual numbers within that range, for example, 1, 2, 2.7, 3, 4, 5, 5.3, and 5. This applies regardless of the breadth of the range.

What is claimed is:

1. A device for detecting photons for tomography, comprising:
a plurality of scintillator arrays, each of the plurality of scintillator arrays configured to emit photons in response to receipt of ionizing radiation, each respective scintillator array of the plurality of scintillator arrays comprising:
a first region corresponding to a first end to an intermedial section of the respective scintillator array, the intermedial section having a first cross-sectional dimension, and
a second region corresponding to the intermedial section to a second end of the respective scintillator array, the second end having a second cross-sectional dimension smaller than the first cross-sectional dimension;
a light guide coupled with the first end of each of the plurality of scintillator arrays, the light guide configured to transfer the photons from a first scintillator array to a second scintillator array of the plurality of scintillator arrays; and
a photodetector coupled with the second end of the second scintillator array, the photodetector configured to convert the photons received via the second scintillator array into an electrical signal for tomography imaging.

2. The device of claim 1, wherein the first region of at least one of the plurality of scintillator arrays further comprises the first end having a third cross-sectional dimension smaller than the first cross-sectional dimension of the intermedial section.

3. The device of claim 1, wherein the second region of at least one of the plurality of scintillator arrays has a third cross-sectional dimension tapering from the first cross-sectional dimension at the intermedial section to a third cross-sectional dimension at the second end, to minimize leakage of photons to a second photodetector neighboring the photodetector.

4. The device of claim 1, wherein at least one of the plurality of scintillator arrays comprises a cuboid structure formed from at least one of a lutetium-based crystal, alkali halide crystal, or an inorganic non-alkali crystal.

5. The device of claim 1, wherein the photodetector has a third cross-sectional dimension corresponding a fourth cross-sectional dimension defined by the second cross-sectional dimension of at least two scintillator arrays of the plurality of scintillator arrays, to localize distribution of photons from the at least two scintillator arrays of the plurality of scintillator arrays.

6. The device of claim 1, wherein the photodetector is further configured to send the electrical signal to a circuit, the circuit configured to perform at least one of time-of-flight (ToF) or depth-of-interaction (DOI) using the electrical signal for tomography imaging.

7. The device of claim 1, wherein the light guide further comprises at least one of a prism structure or a dome structure, the light guide configured to direct photons from the first scintillator array to the second scintillator array of the plurality of scintillator arrays.

8. A system for performing tomographic imaging, comprising:
a block structure having a first layer, a second layer, and a third layer;
a plurality of scintillator arrays arranged along the first layer, each respective scintillator array of the plurality of scintillator arrays comprising:
a first region corresponding to a first end to an intermedial section of the respective scintillator array, the intermedial section having a first cross-sectional dimension, and
a second region corresponding to the intermedial section to a second end of the respective scintillator array, the second region having a second cross-sectional dimension tapering from the first cross-sectional dimension at the intermedial section to a third cross-sectional dimension at the second end, the third cross-sectional dimension smaller than the first cross-sectional dimension;
a plurality of light guides arranged along the second layer, each of the plurality of light guides coupled with at least two of the plurality of scintillator arrays; and
a plurality of photodetectors arranged along the third layer, each of the plurality of photodetectors coupled with at least two of the plurality of scintillator arrays.

9. The system of claim 8, wherein the block structure further comprises an interior region,
wherein at least one of the plurality of light guides in the interior region is coupled with a subset of scintillator arrays including at least four of the plurality of scintillator arrays in the interior region, and
wherein at least one of the plurality of photodetectors in the interior region comprises a prism structure configured to transfer photons from at least one of the subset of scintillator arrays to a remainder of the subset of scintillator arrays.

10. The system of claim 8, wherein the block structure further comprises an edge region,
wherein at least one of the plurality of light guides in the edge region is coupled with a subset of scintillator arrays including a first scintillator array and a second scintillator array of the plurality of scintillator arrays in the edge region, and
wherein at least one of the plurality of photodetectors in the edge region comprises a prism structure configured to transfer photons from the first scintillator array to the second scintillator array.

11. The system of claim 8, wherein the block structure further comprises a corner region,
wherein at least one of the plurality of light guides in the corner region is coupled with a subset of scintillator arrays including at least three of the plurality of scintillator arrays in the corner region, and
wherein at least one of the plurality of photodetectors in the corner region comprises a prism structure configured to transfer photons from a scintillator array from the subset of scintillator arrays on a corner to a remainder of the subset of scintillator arrays.

12. The system of claim 8, wherein the first region of at least one of the plurality of scintillator arrays further comprises the first end having a fourth cross-sectional dimension smaller than the first cross-sectional dimension of the intermedial section.

13. The system of claim 8, the second region of at least one of the plurality of scintillator arrays has a third cross-sectional dimension tapering from the first cross-sectional dimension at the intermedial section to a third cross-sectional dimension at the second end, to minimize leakage of photons to a second photodetector neighboring the photodetector.

14. The system of claim 8, further comprising a circuit coupled with the plurality of photodetectors, the circuit configured to perform at least one of time-of-flight (ToF) or depth-of-interaction (DOI) using a plurality of electrical signals from the plurality of photodetectors.

15. A method of arranging detector blocks for tomography, comprising:
positioning, along a first layer of a block structure, a plurality of scintillator arrays, each respective scintillator array of the plurality of scintillator arrays comprising:
a first region corresponding to a first end to an intermedial section of the respective scintillator array, the intermedial section having a first cross-sectional dimension, and
a second region corresponding to the intermedial section to a second end of the respective scintillator array, the second region having a second cross-sectional dimension tapering from the first cross-sectional dimension at the intermedial section to a third cross-sectional dimension at the second end, the third cross-sectional dimension smaller than the first cross-sectional dimension;
positioning, along a second layer of the block structure, a plurality of light guides, each of the plurality of light guides coupled with at least two of the plurality of scintillator arrays
arranging, along a third layer of the block structure, a plurality of photodetectors, each of the plurality of photodetectors coupled with at least two of the plurality of scintillator arrays.

16. The method of claim 15, further comprising forming the first region of at least one of the plurality of scintillator arrays to have the first end with a third cross-sectional dimension smaller than the first cross-sectional dimension of the intermedial section.

17. The method of claim 15, further comprising forming the second region of at least one of the plurality of scintillator arrays to have a third cross-sectional dimension tapering from the first cross-sectional dimension at the intermedial section to a third cross-sectional dimension at the second end.

18. The method of claim 15, wherein the block structure further comprises an interior region,
wherein at least one of the plurality of light guides in the interior region is coupled with a subset of scintillator arrays including at least four of the plurality of scintillator arrays in the interior region, and
wherein at least one of the plurality of photodetectors in the interior region comprises a prism structure configured to transfer photons from at least one of the subset of scintillator arrays to a remainder of the subset of scintillator arrays.

19. The method of claim 15, wherein the block structure further comprises an edge region,
wherein at least one of the plurality of light guides in the edge region is coupled with a subset of scintillator arrays including a first scintillator array and a second scintillator array of the plurality of scintillator arrays in the edge region, and
wherein at least one of the plurality of photodetectors in the edge region comprises a prism structure configured to transfer photons from the first scintillator array to the second scintillator array.

20. The method of claim 15, wherein the block structure further comprises a corner region,
wherein at least one of the plurality of light guides in the corner region is coupled with a subset of scintillator arrays including at least three of the plurality of scintillator arrays in the corner region, and
wherein at least one of the plurality of photodetectors in the corner region comprises a prism structure configured to transfer photons from a scintillator array from the subset of scintillator arrays on a corner to a remainder of the subset of scintillator arrays.
